(19) United States
(12) Patent Application Publication
(10) Pub. No.: US 2024/0409971 A1
(43) Pub. Date: Dec. 12, 2024

(54) POLYSACCHARIDE OR POLYSACCHARIDE MIXTURE PRODUCED BY PAENIBACILLUS POLYMYXA

(71) Applicant: Technische Universität München, München (DE)

(72) Inventors: Volker Sieber, Nandlstadt (DE); Christoph Schilling, Mannheim (DE); Broder Rühmann, Straubing (DE); Jochen Schmid, Münster (DE)

(21) Appl. No.: 18/690,537

(22) PCT Filed: Aug. 10, 2022
(86) PCT No.: PCT/EP2022/072461

§ 371 Date: Mar. 8, 2024

(30) Foreign Application Priority Data
Sep. 10, 2021 (DE) 10 2021 123 528.6

(51) Int. Cl. C12P 19/04; C12N 9/22; C12N 15/11; C12N 15/90

(52) U.S. Cl. CPC C12P 19/04; C12N 9/22; C12N 15/11; C12N 15/905; C12N 2310/20

(57) ABSTRACT

The invention relates to a polysaccharide or a polysaccharide mixture produced by a genetically modified production organism Paenibacillus polymyxa, as well as to a method for the preparation of the polysaccharide or polysaccharide mixture.

### BACKGROUND

The invention relates to a polysaccharide or a polysaccharide mixture produced by a genetically modified production organism Paenibacillus polymyxa.

Polysaccharides are universal biopolymers that occur in all areas of life. They occur in great abundance and fulfil a variety of tasks in nature. In principle, polysaccharides can be categorised according to their function and/or localisation into intracellular polysaccharides, structural polysaccharides and extracellular polysaccharides. Starch and glycogen are examples of intracellular polysaccharides and are efficient energy storage polysaccharides in animals, algae or plants. Examples of structural polysaccharides are cellulose, chitin, xylan or mannan, which are hard, solid structures that give plants, insects or fungi mechanical strength. Extracellular polysaccharides include all polysaccharides that are secreted into the extracellular space. If the polysaccharides are completely secreted into the extracellular space and do not form an envelope around the cell like capsular polysaccharides, they are also referred to as exopolysaccharides (EPS). However, it is not always possible to clearly distinguish between capsular polysaccharides and exopolysaccharides, as capsular polysaccharides are sometimes only very loosely associated with the cell membrane, and exopolysaccharides can also be located in close proximity to the cell.

All polysaccharides consist of carbohydrate monomers. These monomeric sugars and their modifications form the basic components of polysaccharides. In contrast to their plant counterparts, microbial heteropolysaccharides are usually regularly structured and made up of repeating elements with a consistent monomer sequence—the so-called repeating units. Depending on the species and strain, these repeating units usually consist of two to eight sugar monomers. In extreme cases, repeating units can consist of up to fourteen sugar monomers. The synthesis of these repeating units and their linkage to the polysaccharides are regulated in microorganisms in so-called biosynthesis clusters, in which all the enzymes required for synthesis are usually encoded.

The large number of different monomers and the variety of ways in which they can be combined with each other make countless polymer variants possible. Due to their high structural variability, exopolysaccharides also have very diverse physical and chemical properties. This makes them interesting materials with novel characteristics or additives that give a product the desired properties.

A well-known example of an industrially utilised EPS that has long been established on the market is xanthan gum. It is synthesised by Xanthomonas campestris and is used in food technology as an emulsifier or foam stabiliser, among other things. However, xanthan gum is also used outside the food sector. It is used, for example, to adjust the viscosity of printer inks. Another polysaccharide that has already been launched on the US and EU markets is gellan gum. This is the EPS of the organism Sphingomonas paucimobilis, which is able to form thermoreversible gels and is therefore used in food technology as a gelling agent and stabiliser.

From a biotechnological point of view, such microbial polysaccharides are of particular interest as they can be produced in variable quantities regardless of season and location.

It is known that the Gram-positive soil bacterium Paenibacillus polymyxa produces polysaccharide under suitable fermentation conditions. In particular, Paenibacillus polymyxa is known to produce a polysaccharide that is characterised by its high viscosity. This high viscosity allows the polysaccharide to be used, for example, as a rheological mediator or binder in the food, cosmetics and/or pharmaceutical sectors. On the other hand, the high viscosity of the polysaccharide produced is an obstacle to efficient production of the polysaccharide, as the polysaccharide imparts a high viscosity to the fermentation medium, making mass transport more difficult and requiring increased energy input, for example through stirring or heating.

The object of the invention is to provide an efficient and energy-saving method for the production of a polysaccharide which can be produced by Paenibacillus polymyxa.

The basis for achieving this object was provided by characterising the polysaccharide and clarifying its biosynthesis. It turned out that the polysaccharide produced by Paenibacillus polymyxa is a polysaccharide mixture of three individual polysaccharides, which only exhibits its high viscosity through the interaction of two of these polysaccharides. The biosynthesis of each of these three individual polysaccharides was elucidated by the inventors.

The object of the invention was achieved by mutagenesis, preferably targeted mutagenesis, of individual genes of Paenibacillus polymyxa, which in turn enabled the controlled production of the low-viscosity individual polysaccharides and/or their low-viscosity mixtures. The rheological properties of the polysaccharides and/or the polysaccharide mixtures can be adjusted by mixing in suitable proportions and, for example, also achieve the properties of the polysaccharide mixture produced by the wild type. Accordingly, the invention relates to a production method, as well as to the differently mutated production organisms Paenibacillus polymyxa used in the production method, as well as to the use of the polysaccharides or polysaccharide mixtures produced according to the invention, for example as rheological mediators, binders, stabilising agents, emulsifying agents or flocculating agents, preferably in the food, pharmaceutical and/or cosmetics sector.

### BRIEF DESCRIPTION OF THE DRAWINGS

FIG. 1 shows the structure of the repeating unit of paenan I.

FIG. 2 shows the structure of the repeating unit of paenan II.

FIG. 3 shows the structure of the repeating unit of paenan III.

FIG. 4 shows the monomer composition of the polysaccharides produced. Carbohydrate fingerprints of EPS from P. polymyxa DSM 365 and mutant variants were analysed using the HT-PMP method. Based on the monomer ratios obtained and the detection of specific dimers in the MS analysis, polymer compositions were assigned to the presence of different paenan variants (I-III). The ΔepsO variant shows the same monomer composition as the wild-type polymer, but no pyruvate or the corresponding ketal was detected.

FIG. 5 shows viscosity curves of paenan variants measured at logarithmically increasing shear rates from 0.01 to 1000/s with a cone-plate geometry at 20° C. with (squares □) and without (circles ∘) the addition of 0.5% NaCl. All measurements were carried out in triplicate, error bars show the standard deviation.

FIGS. 6 and 7 show amplitude sweeps of 1% solutions of the indicated EPS variants, produced by P. polymyxa DSM 365, in the aqueous solutions and in the presence of 0.5% NaCl. G′: storage modulus; G″: loss modulus

FIGS. 8 and 9 show frequency sweeps of 1% solutions of the indicated EPS variants, produced by P. polymyxa DSM 365, in the aqueous solutions and in the presence of 0.5% NaCl. Due to a limited LVE range of paenan III under the applied conditions, this variant was not measured in frequency sweeps. G′ (circle): storage modulus; G″ (square): loss modulus

FIGS. 10 and 11 show temperature sweeps from 20-75° C. of 1% solutions of the indicated EPS variants, produced by P. polymyxa DSM 365, in the aqueous solutions and in the presence of 0.5% NaCl. G′ (circle): storage modulus; G″ (square): loss modulus

FIG. 12 shows the monomer composition of the paenan wild type compared to monomer compositions of polysaccharide mixtures that were remixed after separate individual production.

### DESCRIPTION OF THE INVENTION

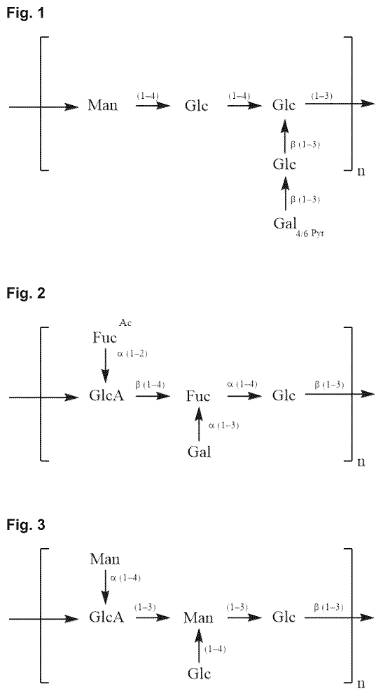

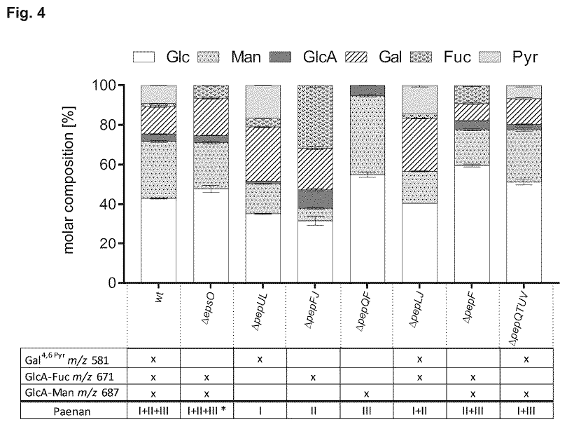

As mentioned above, the inventors discovered that the polysaccharide produced by Paenibacillus polymyxa is a polysaccharide mixture of three individual polysaccharides. These individual polysaccharides are referred to as paenan I, paenan II and paenan III. The structures of the repeating unit of paenan I, paenan II, and paenan III are shown in FIGS. 1, 2, and 3, respectively. The respective molecular weights are shown in Table 4.

The biosynthesis of the individual polysaccharides was elucidated and it was found that the following enzymes are necessary for the production of paenan I: the glycosyltransferases PepD and/or PepF, the pyruvyltransferase EpsO, and the undecaprenyl-glucose-phosphotransferase PepC. Without the glycosyltransferases and the undecaprenyl-glucose-phosphotransferase, the repeating unit is not formed. The pyruvyltransferase EpsO ensures that a pyruvate residue is attached to the terminal repeating unit. This pyruvate residue is essential for the formation of the gel character and the high viscosity of the polysaccharide mixtures produced.

The following enzymes are required for the production of paenan II: the glycosyltransferases PepT, PepU, and/or PepV, and the undecaprenyl-glucose-phosphotransferase PepQ. Without the glycosyltransferases and the undecaprenyl-glucose-phosphotransferase, the repeating unit is not formed. Paenan II also contains a GDP-L-fucose, which is produced by the enzyme GDP-L-fucose synthase. GDP-L-fucose synthase is encoded in the gene fcl, which occurs only once in the Paenibacillus genome. Deletion of fcl can therefore prevent the production of paenan II.

The following enzymes are required for the production of paenan III: the glycosyltransferases PepI, PepJ, PepK, and/or PepL, and the undecaprenyl-glucose phosphotransferases PepC or PepQ. Without the glycosyltransferases and one of the undecaprenyl-glucose phosphotransferases, the repeating unit is not formed.

It was also found that interaction between paenan I and paenan III leads to the formation of the gel character and the high viscosity of the polysaccharide mixtures produced. Here, the pyruvate residue on the repeating unit of paenan I interacts with the glucuronic acid in paenan III. If, after separate production of the individual polysaccharides, a polysaccharide mixture is to be produced which is to achieve the gel character and viscosity of the paenan wild type, then the function of EpsO in the production organism must not be eliminated.

This means that the polysaccharide mixtures of paenan I and paenan II, as well as the polysaccharide mixtures of paenan II and paenan III, also have low viscosity and can be produced simultaneously without the disadvantages of a highly viscous fermentation medium.

These findings now enable mutagenesis, in particular targeted mutagenesis, of the coding sequences for these enzymes, which mutagenesis leads to the loss of enzyme function, and thus the production of production organisms that can specifically produce low-viscosity individual polysaccharides or low-viscosity polysaccharide mixtures. The low-viscosity individual polysaccharides or low-viscosity polysaccharide mixtures can be used, for example, as surface coatings, functional binders for paints, and in the food industry, for example in fruit juices or salad dressings etc.

The desired positive properties of the polysaccharide mixtures, such as adjustable viscosity and gel character, can be restored after separate production by mixing and, if necessary, heating. Thus, the method according to the invention combines the advantages of the efficient production of low-viscosity individual polysaccharides or low-viscosity polysaccharide mixtures with the advantages offered by the high-viscosity polysaccharide mixtures, obtained after mixing the low-viscosity individual polysaccharides or the low-viscosity polysaccharide mixtures, due to their versatile applicability.

The invention thus relates, on the one hand, to a method for producing a polysaccharide or a polysaccharide mixture by a production organism Paenibacillus polymyxa, wherein the method comprises at least one of the following steps separately from one another:
(a) production of the polysaccharide paenan I by a production organism Paenibacillus polymyxa, in which at least one enzymatic function of a polysaccharide biosynthesis cluster is switched off by genetic modification, so that neither the polysaccharide paenan II nor the polysaccharide paenan III is produced; and/or
(b) production of the polysaccharide paenan II by a production organism Paenibacillus polymyxa, in which at least one enzymatic function of a polysaccharide biosynthesis cluster is switched off by genetic modification, so that neither the polysaccharide paenan I nor the polysaccharide paenan III is produced; and/or
(c) production of the polysaccharide paenan III by a production organism Paenibacillus polymyxa, in which at least one enzymatic function of a polysaccharide biosynthesis cluster is switched off by genetic modification, so that neither the polysaccharide paenan I nor the polysaccharide paenan II is produced; and/or
(d) production of the polysaccharides paenan I and II by a production organism Paenibacillus polymyxa, in which at least one enzymatic function of a polysaccharide biosynthesis cluster is switched off by genetic modification, so that the polysaccharide paenan III is not produced; and/or
(e) production of the polysaccharides paenan II and III by a production organism Paenibacillus polymyxa, in which at least one enzymatic function of a polysaccharide biosynthesis cluster is switched off by genetic modification, so that the polysaccharide paenan I is not produced.

The method according to the invention may also comprise purifying the produced polysaccharide or polysaccharide mixture after production.

The production organism that is modified by targeted mutagenesis is Paenibacillus polymyxa. In the embodiment examples, Paenibacillus polymyxa DSM 365 is used. However, all Paenibacillus polymyxa strains which form the polysaccharides paenan I, II, and III under suitable fermentation conditions and/or cultivation conditions known to the person skilled in the art can be used.

The method according to the invention may also comprise, after the preparation and optionally the purification of the polysaccharides or the polysaccharide mixtures, mixing the produced polysaccharides or polysaccharide mixtures, whereby a polysaccharide mixture is obtained, which comprises a mixture of the polysaccharides paenan I and paenan II, or a mixture of the polysaccharides paenan I and paenan III, or a mixture of the polysaccharides paenan II and paenan III, or a mixture of the polysaccharides paenan I, paenan II and paenan III.

As shown in Example 5, the skilled person can select the type of polysaccharides or polysaccharide mixtures to be mixed, the mixing ratio and the concentrations during mixing, and thus set the desired rheological behaviour of the resulting mixture. For example, by mixing paenan I and paenan III in a ratio of 1:2 m/v, a polysaccharide mixture can be obtained that has similar rheological properties to the paenan wild type. However, mixtures can also be produced which, for example, have a higher or lower viscosity compared to the paenan wild type. These properties can be adjusted by the skilled person as required for the respective application.

Methods for specifically switching off the function of a gene are known to those skilled in the art. In the embodiments, the functions of the enzymes described were switched off by CRISPR-Cas9-mediated knock-out of the genetic sequences encoding the respective enzymes. The basic procedure is described in Rütering et al, Synth Biol (Oxf); 2017 January. However, any methods known to the skilled person for specifically switching off the function of a gene can be used. For example, it is not absolutely necessary to delete the entire gene sequence. It is sufficient to delete only the region of the gene sequence that is responsible for the function of the gene. A genetic modification that leads to the elimination of a gene's function can also be obtained through natural mutagenesis, such as mutations caused by UV radiation or chemical agents. The strains mutated in this way can then be selected for the desired phenotype with little effort. Accordingly, genetic modification within the meaning of the present invention is not limited to genetic engineering.

In order to obtain production organisms according to the invention, it may be sufficient to switch off only one function of a gene, as long as the switching off of this gene results in the undesired individual polysaccharide no longer being produced by the mutant production organism. Specific embodiments with exemplary mutations are listed in the examples.

The method according to the invention may comprise that the genetic modification, by which at least one enzymatic function of a polysaccharide biosynthesis cluster is switched off so that the polysaccharide paenan I cannot be produced, is selected from genetic modifications which suppress the function of the glycosyltransferase PepD and/or PepF, and/or the undecaprenyl-glucose-phosphotransferase PepC.

The method according to the invention may alternatively or additionally comprise that the genetic modification, by which at least one enzymatic function of a polysaccharide biosynthesis cluster is switched off so that the polysaccharide paenan II cannot be produced, is selected from genetic modifications which suppress the function of the glycosyltransferase PepT, PepU, and/or PepV, and/or the undecaprenyl-glucose-phosphotransferase PepQ, and/or the GDP-L-fucose synthase.

The method according to the invention may alternatively or additionally comprise that the genetic modification, by which at least one enzymatic function of a polysaccharide biosynthesis cluster is switched off so that the polysaccharide paenan III cannot be produced, is selected from genetic modifications which suppress the function of the glycosyltransferase PepI, PepJ, PepK, and/or PepL.

Exemplary genetic modifications which switch off at least one enzymatic function of a polysaccharide biosynthesis cluster, so that neither the polysaccharide paenan II nor the polysaccharide paenan III is produced, are selected from genetic modifications which switch off the function of the GDP-L-fucose synthase or the function of the glycosyltransferases PepI, PepT, PepU, and PepV, or the function of the glycosyltransferases PepK, PepT, PepU, and PepV, or the function of the glycosyltransferases PepL, PepT, PepU, and PepV, or the function of the glycosyltransferases PepT and PepL.

Exemplary genetic modifications which switch off at least one enzymatic function of a polysaccharide biosynthesis cluster, so that neither the polysaccharide paenan I nor the polysaccharide paenan III are produced, are selected from genetic modifications which suppress the function of the glycosyltransferases PepF and PepJ, or the function of the glycosyltransferases PepD and PepJ.

Exemplary genetic modifications which switch off at least one enzymatic function of a polysaccharide biosynthesis cluster, so that neither the polysaccharide paenan I nor the polysaccharide paenan II are produced, are selected from genetic modifications which suppress the function of the undecaprenyl-glucose phosphotransferase PepQ and the glycosyltransferase PepF.

The invention also relates to a composition comprising the polysaccharide paenan I, but neither the polysaccharide paenan II nor the polysaccharide paenan III; or
a composition comprising the polysaccharide paenan II but neither the polysaccharide paenan I nor the polysaccharide paenan III; or
a composition comprising the polysaccharide paenan III but neither the polysaccharide paenan I nor the polysaccharide paenan II; or
a composition comprising the polysaccharide paenan I and the polysaccharide paenan II but not the polysaccharide paenan III; or
a composition comprising the polysaccharide paenan II and the polysaccharide paenan III but not the polysaccharide paenan I.

The invention also relates to a production organism Paenibacillus polymyxa, in which at least one enzymatic function of a polysaccharide biosynthesis cluster is switched off by genetic modification, so that neither the polysaccharide paenan II nor the polysaccharide paenan III can be produced; or
a production organism Paenibacillus polymyxa, in which at least one enzymatic function of a polysaccharide biosynthesis cluster is switched off by genetic modification, so that neither the polysaccharide paenan I nor the polysaccharide paenan III can be produced; or
a production organism Paenibacillus polymyxa, in which at least one enzymatic function of a polysaccharide biosynthesis cluster is switched off by genetic modification, so that neither the polysaccharide paenan I nor the polysaccharide paenan II can be produced; or
a production organism Paenibacillus polymyxa, in which at least one enzymatic function of a polysaccharide biosynthesis cluster is switched off by genetic modification, so that the polysaccharide paenan III cannot be produced; or
a production organism Paenibacillus polymyxa, in which at least one enzymatic function of a polysaccharide biosynthesis cluster is switched off by genetic modification, so that the polysaccharide paenan I cannot be produced.

The polysaccharides or polysaccharide mixtures produced with the method according to the invention by the production organisms according to the invention can be used in a variety of ways due to their advantageous properties, which on the one hand can already be present when a polysaccharide produced according to the invention or a polysaccharide mixture produced according to the invention is used per se, or can be obtained when a polysaccharide produced according to the invention or a polysaccharide mixture produced according to the invention is mixed with a further polysaccharide produced according to the invention or a further polysaccharide mixture produced according to the invention.

Accordingly, the present invention also comprises the use of a polysaccharide comprising paenan I, paenan II, or paenan III, or a mixture of any combination of paenan I, paenan II, and paenan III, as a rheological mediator, binder, stabilising agent, emulsifying agent, or flocculating agent, preferably in the food, pharmaceutical, and/or cosmetics sector.

In particular, the polysaccharide comprising paenan I, paenan II, or paenan III, or a mixture of any combination of paenan I, paenan II, and paenan III, may be used as an additive to a food product, for example, and without being limited thereto, from bakery products, soups, sauces, mayonnaise, ketchup, jams, marmalades, jellies, canned foods (fruit and vegetables), salad dressing, ice cream, milk-based mixed drinks, puddings, pickles, tinned meat and fish, or as an additive in a cosmetic product, for example, and without being limited thereto, selected from a shower gel, cosmetic creams and lotions, hair shampoo, skin creams, toothpaste, moisturisers, or as an additive in a pharmaceutical or medical product, for example, and without being limited thereto, selected from wound dressings, controlled release particles, eye drops, tablet coatings, capsules for food supplements, or for use in tissue engineering to support surface adhesion after surgery, in water flooding for petroleum recovery, in bioremediation and wastewater treatment, for heavy metal binding, as a cement additive to keep particles in suspension during curing, or as a paint additive.

The method according to the invention, the production organisms according to the invention and the uses according to the invention will now be explained by way of example below.

### EXAMPLES

### Example 1—Production of the Mutant Production Organisms

P. polymyxa DSM 365 was obtained from the Deutsche Sammlung für Mikroorganismen und Zellkultur (DSMZ, Germany). Escherichia coli NEB Turbo cells (New England Biolabs, USA) were used for the plasmid constructions. E. coli S17-1 (DSMZ strain DSM 9079) was used to transform P. polymyxa DSM 365 via conjugation. The strains produced are listed in Table 1.

TABLE 1
Overview of the manufactured production organisms

| manufactured | experimentally generated | basic rheological |
|---|---|---|
| paenan | combinations | properties |
| I | ΔpepITUV, ΔpepKTUV, | low viscosity, aqueous |
| | ΔpepLTUV, ΔpepTL | |
| II | ΔpepFJ, ΔpepDJ | low viscosity, aqueous |
| III | ΔpepQF | low viscosity, aqueous |
| I + II | ΔpepI, ΔpepJ, ΔpepK, ΔpepL | low viscosity, aqueous |
| II + III | ΔpepC, ΔpepF, ΔpepD, ΔpepCF | low viscosity |
| I + III | ΔpepQ, ΔpepT, ΔpepU, ΔpepV, | high viscosity, |
| | ΔpepTUV, ΔpepQTUV | gelling agent |
| I + II + III | ΔepsO | low viscosity, |
| depyruvylated | | creamy |
| I + II + III | Wild type | high viscosity, |
| (wt) | P. polymyxa DSM 365 | gelling agent |

All knock-outs were performed as previously described (Rütering et al., 2017). In brief, gRNAs for each target were cloned into the plasmid pCasPP via Golden Gate assembly using BbsI. Subsequently, 1 kb upstream and downstream homology flanks of the gene of interest were ligated into a unique SpeI site, followed by transformation of chemically competent E. coli S17-1. P. polymyxa was transformed by conjugation using E. coli S17-1 harbouring the different plasmids. Overnight cultures of donor and recipient strains were diluted 1:100 with selective or non-selective LB media and cultured at 37° C. for 3 h at 280 rpm. 900 μl of the recipient culture was subjected to heat shock at 42° C. for 15 min and mixed with 300 μl of the donor strain. The cells were centrifuged for 2 min at 6,000×g, resuspended in 800 μl LB medium and dropped onto non-selective LB agar plates. After 24 h incubation at 30° C., the cells were scraped off, resuspended in 500 μl LB broth and 100 μl of this was plated on selective LB agar containing 50 μg/ml neomycin and 20 μg/ml polymyxin for counter-selection. P. polymyxa conjugants were analysed for successful transformation by colony PCR after incubation at 30° C. for 48 hours. Confirmed knock-out strains were plasmid-cured by cultivation in LB broth without antibiotic selection pressure and subsequent replica plating on LB agar plates both with and without neomycin. Strains that did not grow on plates with selection markers were verified by sequencing the target region and used for further experiments. All plasmids and oligonucleotides used to obtain knock-out strains are listed in Tables 2 and 3 below

TABLE 2
Some of the plasmids used and produced.

| Plasmid | Description |
|---|---|
| pCasPPH_pepC | knock-out plasmid directed against pepC with repair template |
| pCasPPH_pepD | knock-out plasmid directed against pepD with repair template |
| pCasPPH_pepF | knock-out plasmid directed against pepF with repair template |
| pCasPPH_pepI | knock-out plasmid directed against pepI with repair template |
| pCasPPH_pepJ | knock-out plasmid directed against pepJ with repair template |
| pCasPPH_pepK | knock-out plasmid directed against pepK with repair template |
| pCasPPH_pepL | knock-out plasmid directed against pepL with repair template |
| pCasPPH_pepQ | knock-out plasmid directed against pepQ with repair template |
| pCasPPH_pepT | knock-out plasmid directed against pepT with repair template |
| pCasPPH_pepU | knock-out plasmid directed against pepU with repair template |
| pCasPPH_pepV | knock-out plasmid directed against pepV with repair template |
| pCasPPH_epsO | knock-out plasmid directed against epsO with repair template |
| pCasPPH_fcI | knock-out plasmid directed against fcI with repair template |

TABLE 3
Some of the oligonucleotides used. Overhangs used for the Golden Gate
assembly are shown in lower case. Restriction sites used for cloning
are underlined. For each KO construct, two sets of sgRNAs were designed,
tested and listed below when successful knock-outs were achieved

| Primer | Sequence 5′→3′ |
|---|---|
| pepC_sgRNA1_fw | acgcGAGCATGGAAAATTTACCGG (SEQ ID NO: 01) |
| pepC_sgRNA1_rev | aaacCCGGTAAATTTTCCATGCTC (SEQ ID NO: 02) |
| pepC_sgRNA2_fw | aaacCCTTGATATGCTGCTGTCGT (SEQ ID NO: 03) |
| pepC_sgRNA_2_rev | acgcACGACAGCAGCATATCAAGG (SEQ ID NO: 04) |
| pepC_Harms_US_fw | GGCCTCTAGACCAAAATTCGATGAAGCAAGGAGAATGAGTGCG (SEQ ID NO: 05) |
| pepC_Harms_US_rev_OE | CGCTTGAATGCATCCATCAATAAGCATCCCGTACCCCCTCTCAACCTGCCGTGG (SEQ ID NO: 06) |
| pepC_Harms_DS_fw_OE | CCACGGCAGGTTGAGAGGGGGTACGGGATGCTTATTGATGGATGCATTCAAGCG (SEQ ID NO: 07) |
| pepC_Harms_DS_rev | GGCCTCTAGACCATCTTTCACCCCGCAGCTGACTCG (SEQ ID NO: 08) |
| pepC_KO_proof_fw | GCAACTCATGGAGCAGGCCAGTGCGACG (SEQ ID NO: 09) |
| pepC_KO_proof_rev | GCTGTATGGTGTTTATTCTAGTAGTCCAAGG (SEQ ID NO: 10) |
| pepD_sgRNA1_fw | acgcCAAAGTGTTATACATCACGG (SEQ ID NO: 11) |
| pepD_sgRNA1_rev | aaacCCGTGATGTATAACACTTTG (SEQ ID NO: 12) |
| pepD_sgRNA2_fw | acgcATGAAGCGGGAACATAACGG (SEQ ID NO: 13) |
| pepD_sgRNA_2_rev | aaacCCGTTATGTTCCCGCTTCAT (SEQ ID NO: 14) |
| pepD_Harms_US_fw | CCGGTCTAGAGGCTATTATGGGTCGCTACGTG (SEQ ID NO: 15) |
| pepD_Harms_US_rev_OE | AGTCCAAGGATAGTTACTCATGTTOCCAATAAGCATCCTTGGAGAACAGC (SEQ ID NO: 16) |
| pepD_Harms_DS_fw_OE | TTCTCCAAGGATGCTTATTGGGAACATGAGTAACTATCCTTGGACT (SEQ ID NO: 17) |
| pepD_Harms_DS_rev | AATTTCTAGACGTTACCGCGCCAAGACCTC (SEQ ID NO: 18) |
| pepD_KO_proof_fw | CCGGATCAGCAGGACGGACG (SEQ ID NO: 19) |
| pepD_KO_proof_rev | GCCCGCAACCGATATATCCC (SEQ ID NO: 20) |
| pepI_sgRNA1_fw | acgcAGCATTATGAGCTAACCAAG (SEQ ID NO: 21) |
| pepI_sgRNA1_rev | aaacCTTGGTTAGCTCATAATGCT (SEQ ID NO: 22) |
| pepI_Harms_US_fw | aattTCTAGAGGCTGCGCTCTTTTTCTTCATC (SEQ ID NO: 23) |
| pepI_Harms_US_rev_OE | GCAATACGAAATCCGTAGCGGGTGCCTCCTTCTTTAT (SEQ ID NO: 24) |
| pepI_Harms_DS_fw_OE | AGGAGGCACCCGCTACGGATTTCGTATTGCGGCAAC (SEQ ID NO: 25) |
| pepI_Harms_DS_rev | ttaaTCTAGAGGCTTCACTGTCCGTTCCCT (SEQ ID NO: 26) |
| pepI_KO_proof_fw | GTTGCTGGCTACCATGTTCGG (SEQ ID NO: 27) |
| pepI_KO_proof_rev | CCTTGCATTCGGTAGAAGTTCACG (SEQ ID NO: 28) |
| pepK_sgRNA2_fw | acgcACAATCGGCTTCAGATACGG (SEQ ID NO: 29) |
| pepK_sgRNA_2_rev | aaacCCGTATCTGAAGCCGATTGT (SEQ ID NO: 30) |
| pepK_Harms_US_fw | aattTCTAGACATACGATTGGCAGCAAGCAG (SEQ ID NO: 31) |
| pepK_Harms_US_rev_OE | TTTAACGCGAATGTCGGTTTCGCCGCCGCCTTTCTGTCA (SEQ ID NO: 32) |
| pepK_Harms_DS_fw_OE | AAAGGGGGGGGCGAACCGACATTCGCGTTAAAGGATGAT (SEQ ID NO: 33) |
| pepK_Harms_DS_rev | AATTTCTAGAGCTGCTCGACCTTTGCGACGGC (SEQ ID NO: 34) |
| pepK_KO_proof_fw | GCTCTAATCTGTAGTGACGAGCAG (SEQ ID NO: 35) |
| pepK_KO_proof_rev | CATCAAATTTGCGGGCATGGG (SEQ ID NO: 36) |
| pepL_sgRNA1_fw | acgcGGAGGCTCCTATCTTCACAA (SEQ ID NO: 37) |
| pepL_sgRNA1_rev | aaacTTGTGAAGATAGGAGCCTCC (SEQ ID NO: 38) |
| pepL_sgRNA2_fw | acgcGTTATTGTCACACACCGATG (SEQ ID NO: 39) |
| pepL_sgRNA_2_rev | aaacCATCGGTGTGTGACAATAAC (SEQ ID NO: 40) |
| pepL_Harms_US_fw | AATTTCTAGAGCGCATCGTTCCATTGGACG (SEQ ID NO: 41) |
| pepL_Harms_US_rev_OE | CCAGCTTCATTCCGTCTAAATCATCCTTTAACGCGAATGTCGG (SEQ ID NO: 42) |
| pepL_Harms_DS_fw_OE | TAAAGGATGATTTAGACGGAATGAAGCTGGCTGTAATT (SEQ ID NO: 43) |
| pepL_Harms_DS_rev | AATTTCTAGAGCGCCTGCCTGAATGAGTGT (SEQ ID NO: 44) |
| pepL_KO_proof_fw | CGGAGAACATATCGACCCGCTC (SEQ ID NO: 45) |
| pepL_KO_proof_rev | GGCCAGTTGTACGAGATCGACC (SEQ ID NO: 46) |
| pepT_sgRNA1_fw | acgcAGAACGTGAGGAAAACCGTG (SEQ ID NO: 47) |
| pepT_sgRNA1_rev | aaacCACGGTTTTCCTCACGTTCT (SEQ ID NO: 48) |
| pepT_sgRNA2_fw | acgcTATGTCGTGGGATCGCATTG (SEQ ID NO: 49) |
| pepT_sgRNA_2_rev | aaacCAATGCGATCCCACGACATA (SEQ ID NO: 50) |
| pepT_Harms_US_fw | AATTTCTAGACCCGGATCATCGCTTTACAGT (SEQ ID NO: 51) |
| pepT_Harms_US_rev_OE | TGCTGACCTGTTGGCGGGGTTTAGAGACCGCTGCG (SEQ ID NO: 52) |
| pepT_Harms_DS_fw_OE | CTGAAGAAGCGCTAGGCCAACAGGTCAGCAGACAG (SEQ ID NO: 53) |
| pepT_Harms_DS_rev | AATTTCTAGAGTCCTCCACGACTTCCTCCAG (SEQ ID NO: 54) |
| pepT_KO_proof_fw | CGCAGCTCAAACGTGGGCTA (SEQ ID NO: 55) |
| pepT_KO_proof_rev | GGGCTACTGCACAGCGAATC (SEQ ID NO: 56) |
| pepU_sgRNA1_fw | acgcAGAAACCGAACATCTTCCTG (SEQ ID NO: 57) |
| pepU_sgRNA1_rev | aaacCAGGAAGATGTTCGGTTTCT (SEQ ID NO: 58) |
| pepU_sgRNA2_fw | acgcTTTACCGCGATACGATCCAG (SEQ ID NO: 59) |
| pepU_sgRNA_2_rev | aaacCTGGATCGTATCGCGGTAAA (SEQ ID NO: 60) |
| pepU_Harms_US_fw | AATTTCTAGAGGATGCTTATGGTCTGGTCATTACT (SEQ ID NO: 61) |
| pepU_Harms_US_rev_OE | CACATTCACTTCCTGTCTGCTGACCTGTTGGC (SEQ ID NO: 62) |
| pepU_Harms_DS_fw_OE | CAGCAGACAGGAAGTGAATGTGAAGCAGCTGAT (SEQ ID NO: 63) |
| pepU_Harms_DS_rev | AATTTCTAGAGCACGCAGCTGATGAATTTTATCC (SEQ ID NO: 64) |
| pepU_KO_proof_fw | GCGTATTATCTGGCTCCACGG (SEQ ID NO: 65) |
| pepU_KO_proof_rev | GTTGTAAAGCTTAGTCGGCCTTGT (SEQ ID NO: 66) |
| pepV_sgRNA1_fw | acgcTTACCCTGGAATCCGCATCG (SEQ ID NO: 67) |
| pepV_sgRNA1_rev | aaacCGATGCGGATTCCAGGGTAA (SEQ ID NO: 68) |
| pepV_Harms_US_fw | aattTCTAGAAAGAATCAAGAAGCTGCTTTTACAGG (SEQ ID NO: 69) |
| pepV_Harms_US_rev_OE | TTTACCGACTGCATCCGCAATCCTTCCAACT (SEQ ID NO: 70) |
| pepV_Harms_DS_fw_OE | ATTGCGGATGCAGTCGGTAAAAGCAGGATGCA (SEQ ID NO: 71) |
| pepV_Harms_DS_rev | aattTCTAGAGCCAGTCCCCATATACCAATCG (SEQ ID NO: 72) |
| pepV_KO_proof_fw | AGCGAACATGGCAAAGCTGG (SEQ ID NO: 73) |
| pepV_KO_proof_rev | GGGGCATCCCTTACGTCATCT (SEQ ID NO: 74) |
| fcI_sgRNA1_fw | acgcGATACATCTACCAACTTACG (SEQ ID NO: 75) |
| fcI_sgRNA1_rev | aaacCGTAAGTTGGTAGATGTATC (SEQ ID NO: 76) |
| fcI_sgRNA2_fw | acgcACAAACGAATGTGATTGATG (SEQ ID NO: 77) |
| fcI_sgRNA2_rev | aaacCATCAATCACATTCGTTTGT (SEQ ID NO: 78) |
| fcI_US_harms_fw | AATTAATCTAGAGACGTGATCTTAATGGATACGCCG (SEQ ID NO: 79) |
| fcI_US_harms_rev_OE | CCACCGTTTTCTTTAATACGCATCCTTGGAGCCT (SEQ ID NO: 80) |
| fcI_DS_harms_fw_OE | CGTATTAAAGAAAACGGTGGGGGAATTGG (SEQ ID NO: 81) |
| fcI_DS_harms_rev | AATTTCTAGATCTCTTACACGACGACAGAAGC (SEQ ID NO: 82) |
| fcI_Koproof_fw | AATCCGTCCGAGATGCTCAG (SEQ ID NO: 83) |
| fcI_Koproof_rev | CCTTGCGTACAGCGAAGAGA (SEQ ID NO: 84) |

### Example 2—Fermentative Production of Polysaccharides

All media components were obtained from Carl Roth GmbH (Germany), unless otherwise stated. For cloning procedures, strains were grown in LB media (5 g/l yeast extract, 10 g/l tryptone, 10 g/l NaCl) and additionally with 50 μg/ml neomycin and 20 μg/ml polymyxin. Supplemented if necessary. All strains were stored in 30% glycerol at −80° C. Before cultivation, the strains were spread on LB agar plates and allowed to grow for 24 h at 30° C.

The fermentation medium contained 30 g/l glucose, 0.05 g/l CaCl₂)×2H₂O, 5 g/l tryptone, 1.33 g/l MgSO₄×7H₂O, 1.67 g/l KH₂PO₄, 2 ml/l RPMI 1640 vitamin solution (Merck, Germany) and 1 ml/l trace element solution (2.5 g/l FeSO₄, 2.1 g/l C₄H₄O₆Na₂×2H₂O, 1.8 g/l MnCl₂×4H₂O, 0.258 g/l H₃BO₃, 0.031 g/l CuSO₄×5H₂O, 0.023 g/l NaMoO₄×2H₂O, 0.075 g/l CoCl₂×7H₂O, 0.021 g/l ZnCl₂). The preculture medium was prepared in the same way as the fermentation medium, except for a reduced glucose concentration of 10 g/l and an additional 20 g/l MOPS, buffered to pH 7.

The fermentative production of EPS was carried out in 1 L Benchtop DASGIP parallel bioreactor systems (Eppendorf, Germany) with a working volume of 500 ml, equipped with a 6-blade Rushton impeller for 28 h with a controlled pH of 6.8 and a pO₂ saturation of 30%. Batch cultivations were started with an initial OD₆₀₀ of 0.1 by inoculation with a suitable volume of preculture. After fermentation, the biomass was separated by centrifugation (15,000×g, 20° C., 20 min), followed by cross-flow filtration of the supernatant using a 100 kDa filtration cassette (Hydrosart, Sartorius AG, Germany). Highly viscous EPS variants, such as those produced from the wild type, were diluted 1:10 with ddH₂O before centrifugation. The concentrated supernatant was then slowly poured into two volumes of isopropanol. Precipitated EPS was collected and dried overnight in a VDL53 vacuum oven at 40° C. (Binder, Germany). The dry weight of the EPS obtained was determined gravimetrically before the EPS was ground to a fine powder in a ball mill at 30 Hz for 1 min (Mixer Mill MM400, Retsch GmbH, Germany).

### Example 3—Characterisation of the Polysaccharides Produced

The monomer composition of manufactured EPS variants was analysed using the 1-phenyl-3-methyl-5-pyrazolone high throughput method (HT-PMP) (Rühmann, Schmid & Sieber, 2014). In brief, 0.1% EPS solutions were hydrolysed in a 96-well plate, sealed with a silicone mat and further covered with 2 M TFA (90 min, 121° C.) using a custom-made metal device. Samples were neutralised with 3.2% NH₄OH. 75 μl PMP master mix (0.1 M methanolic PMP: 0.4% ammonium hydroxide 2:1) was added to 25 μl neutralised hydrolysate and incubated for 100 min at 70° C. in a thermocycler. 20 μl of the derivatised samples were mixed with 25 μl 0.5 M acetic acid and 125 μl ddH₂O and filtered with a 0.2 μm filter plate (1,000×g, 2 min), followed by HPLC-UV-MS using an Ultimate 3000 RS HPLC system (Dionex, USA). The separation was carried out on a reversed phase column (Gravity C18, 100×2 mm, 1.8 μm particle size, Macherey-Nagel, USA), which was set to 50° C. Gradient elution was performed using a mobile phase A (5 mM ammonium acetate (pH 5.6) with 15% acetonitrile) and a mobile phase B (100% acetonitrile) at a constant pump rate of 0.6 ml/min.

To detect the presence of individual paenan polymers, the carbohydrate fingerprint was determined for each variant (FIG. 4). A monomer ratio of 3:1:1:1 (Glc:Man:Gal:Pyr) was expected for paenan I, while an equimolar ratio of Glc:Gal:GlcA:Fuc was expected for paenan II and a monomer ratio of 2:2:1 (Glc:Man:GlcA) for paenan III. Due to the high susceptibility of uronic acids to degradation during chemical hydrolysis, GlcA was strongly underestimated for all polysaccharide compositions containing uronic acid. In addition to the monomer composition, previously identified key dimers detected by MS/MS analysis were used to assign combinatorial knock-out variants to each paenan variant. By assigning the presence of pyruvate ketals to paenan I, a GlcA-Fuc dimer to paenan II and a GlcA-Man dimer to paenan III, the polymer composition of each knock-out variant was determined while maintaining the natural polysaccharide distribution in these variants. From now on, each EPS variant will be named after the paenan variants present and not after the gene deletions that lead to the respective phenotype.

The molecular weight of polymer variants was determined by size exclusion chromatography using an Agilent 1260 Infinity system (Agilent Technologies, Germany) equipped with a refractive index detector (SECcurity GPC1260) and a SECcurity SLD7000 static seven-angle light scattering detector (PSS Polymer Standards Service, Germany). For this purpose, 0.5 g/l of each variant was reconstituted in 0.1 M LiNO3 and 100 μl of sample was injected into the system at 30 min intervals and maintained at 50° C. with a TSKgel SuperMP (PW)-H precolumn and two consecutive TSKGel SuperMultipore PW-H columns (6.0 mm ID×15 cm, TOSOH Bioscience, Germany). The eluent used was 0.1 M LiNO₃ at a constant flow rate of 0.3 ml/min. The absolute molecular weight was determined by light scattering and polymer concentration and further cross-validated with a 12-point pullulan standard (384 Da-2.35 MDa) and a 4.5 MDa xanthan gum reference (Table 4).

TABLE 4
Calculated molecular weights of paenan variants obtained
by GPC analysis using 0.5% EPS solutions in 0.1M LiNO₃.

| Paenan variant | Molecular weight |
|---|---|
| I & II & III | 2.6 · 10⁶ Da |
| I & II & III | 8.8 · 10⁶ Da |
| depyruvylated | |
| I | 5.8 · 10⁶ Da |
| II | 5.5 · 10⁵ Da |
| III | 3.9 · 10⁶ Da |
| I & II | 2.8 · 10⁶ Da |
| I & III | 2.8 · 10⁶ Da |
| II & III | 2.7 · 10⁶ Da |
| Xanthan gum | 4.5 · 10⁶ Da |
| reference | |

Despite the presence of three different polymers in the wild-type EPS, no clear separation of the individual paenan variants was possible. The analysis of individual paenan variants revealed a similar molecular weight distribution for paenan I and paenan III. Only paenan II appears to be significantly smaller with a size of 5·5·10⁵ Da. Given the low proportion of paenan II in the wild-type polymer, this may explain why previous attempts to analyse the heteroexopolysaccharide of P. polymyxa DSM 365 failed to distinguish between several paenan variants. Interestingly, although the depyruvylated polymer also showed a small peak at about 3.0·10⁶ Da, the main molar mass was detected to be significantly larger at 8.8·10⁶ Da compared to all other paenan variants. Compared to the production of xanthan gum, in which the side chains are irregularly provided with acetyl or pyruvyl residues, all the repeating units of paenan I appear to be modified with a pyruvate ketal. Consequently, the loss of this feature in the ΔepsO knockout variant could affect chain length control in P. polymyxa, resulting in increased molecular weight and different rheological behaviour. Alternatively, pyruvylation can also influence the hydrodynamic radius of the polymer and thus the SEC-MALS analysis.

### Example 4—Rheological Properties

For rheological analysis, 1% (w/w) solutions of each polymer were prepared in ddH₂O or 0.5% NaCl (85 mM). The conductivity of each solution was measured using an LF413T ID electrode (Schott Instruments, Germany) to determine the residual salt concentrations of the fermentation medium. Rheological measurements were carried out using an MCR 300 stress-controlled rotational rheometer (Anton Paar, Austria) equipped with a CP 50-1 cone-plate measuring system (50 mm diameter, 1° cone angle, 50 μm measuring gap). All measurements, with the exception of temperature sweeps, were performed at 20° C. controlled by a TEK 150P temperature unit. After applying the solution to the rheometer, all samples were incubated at 20° C. for 5 minutes before starting the measurements. All experiments were carried out in technical triplicates.

Viscosity curves were measured with a logarithmically increased shear rate from 10⁻³ to 10³/s by measuring 3 data points per decade with decreasing measurement time of 100−5 s per data point.

Amplitude sweeps were measured with a logarithmically increasing shear stress amplitude of 10⁻¹ to 10³ Pa at a frequency of 1 Hz.

Frequency sweeps were performed in the linear viscoelastic range (LVE) with a logarithmically increasing frequency of 10⁻² to 10 Hz.

Temperature sweeps were performed within the LVE at a frequency of 1 Hz, applying a temperature rise from 20 to 75° C. at a heating rate of 4° C./min. The edge of the cone-plate measuring system was covered with low-viscosity paraffin oil (Carl Roth, Germany) to prevent evaporation.

The thixotropic behaviour was evaluated by a three-stage oscillatory shear sequence. In the first stage, the samples were subjected to a shear stress within the LVE range, followed by a high oscillatory shear of 10³ Pa for 30 s. The structural recovery was then measured over 10 min within the LVE.

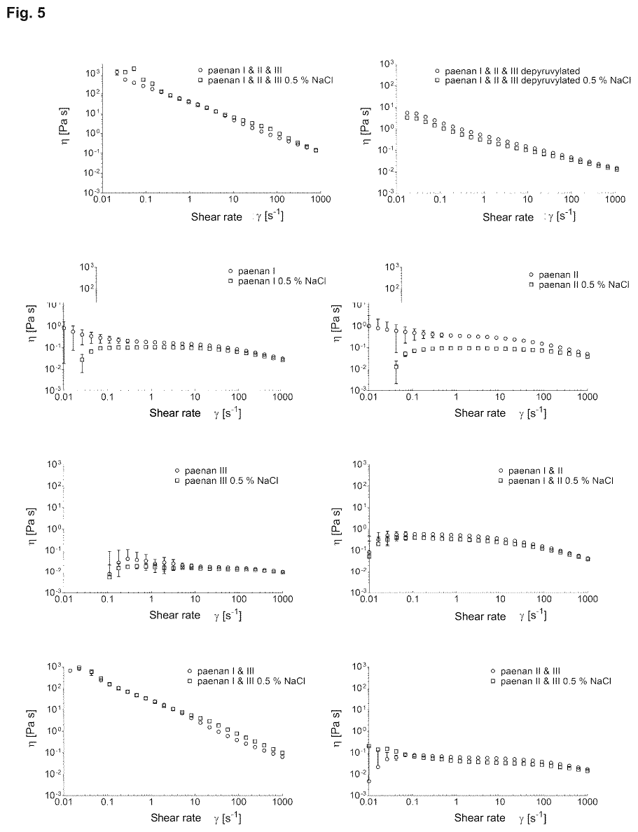

Analyses of the flow behaviour showed a general shear thinning behaviour of all paenan variants (FIG. 5). The highest viscosities and shear thinning behaviour were observed for paenan wild type (I & II & III) with addition of NaCl, followed by paenan wild type without monovalent cations. All individual variants of paenan I-III showed no significantly high viscosities and almost Newtonian flow behaviour in the medium shear rate ranges. Only paenan wild type and paenan I & III showed a strictly shear-thinning behaviour according to the power law and high viscosities, which increased in the presence of 0.5% NaCl. During the depyruvylation of paenan wild type, the viscosity decreased drastically and the addition of NaCl now had the detrimental effect comparable to the depyruvylation of xanthan gum. In the case of xanthan gum, the effect is due to intermolecular interactions between individual polymer molecules mediated by lower cations. The data of the different paenan polymers suggest a similar effect between the paenan I and paenan III molecules. The combination of paenan I & III showed almost the same properties as the combination of paenan I & II & III, which underscores the interaction between paenan I and III as the main reason for the high viscosity of the polymers. This interaction can be explained by the cation-mediated interaction of the terminal pyruvate residue of paenan I with the negative charge of the glucuronic acid in the backbone of paenan III. Interestingly, both combinations of paenan I & II and paenan II & III did not lead to the behaviour of the wild type. Consequently, the glucuronic acid in the backbone of paenan II did not appear to interact with the pyruvyl residue of paenan I, as shown by the low viscosity of this combination. This is particularly interesting as the individual side chain of paenan II would suggest a better accessibility of the charge in paenan II compared to the two side chains of paenan III bound to the glucuronic acid and the neighbouring mannose (FIGS. 1-3). There are two explanations for the interactions between the individual molecules. Firstly, the formation of individual homohelices of paenan I or II or III molecules and a subsequent interaction of these helices. Secondly, the formation of heterohelices of paenan I & II & III. The analysis of the individual polymers provides evidence in favour of the latter. The formation of homohelices would also indicate strong interactions between those of paenan I alone if, as suspected, the pyruvylated side chain protrudes outwards, as in xanthan gum. On the other hand, if paenan I were to form homohelices, an inwardly projecting side chain would lead to a structure similar to that described for diutane gum and could also be the reason for the reduced viscosity. In contrast to diutane gum, the main chain does not carry a negative charge due to the absence of a uronic acid in the backbone of paenan I. In all cases, differences in the helical arrangement of paenan II and paenan III could explain the strong interactions of paenan I & III, but not between paenan I & II and between paenan II & III, which prompted further investigation of the secondary and tertiary polymer structures.

A detailed examination of the individual polymer variants and the combination of paenan II & III revealed several structurally viscous regions, which are indicated by up to three different K and n values of the power laws of the individual sections (Table 5). This phenomenon was observed for paenan I and paenan II individually as well as for combinations of paenan I & II and paenan I & III. However, for paenan III, paenan I & III or the wild-type composition, only a single structurally viscous region was observed. In paenan I & II, the Newton region is more pronounced in the presence of NaCl, which could be the reason for the slight onset of a Newton region in paenan wild-type and paenan I & III in the presence of NaCl. Consequently, this effect could be attributed to paenan I or paenan II.

TABLE 5
Model parameters (K, n) of the power law adjustments of the
various paenan combinations with and without the addition of
0.5% NaCl. If several sections have been adjusted individually,
the K and n values of the individual section are displayed
in ascending order of the corresponding shear rate range.

| Paenan variant | Solution | K | n |
|---|---|---|---|
| Wild type (wt) | aq. | 29.02 | 0.152 |
| (I & II & III) | 0.5% NaCl | 34.38 | 0.171 |
| wt (I & II & III) | aq. | 0.47 | 0.485 |
| depyruvylated | 0.5% NaCl | 0.29 | 0.534 |
| I & II | aq. | 0.01 | 0.830 |
| | 0.5% NaCl | 0.01 | 0.850 |
| I & III | aq. | 24.80 | 0.114 |
| | 0.5% NaCl | 27.41 | 0.203 |
| II & III | aq. | 0.06/0.16 | 0.924/0.679 |
| | 0.5% NaCl | 0.03/0.05/0.10 | 0.677/0.890/0.718 |
| I | aq. | 0.05/0.18/0.52 | 0.390/0.898/0.597 |
| | 0.5% NaCl | 0.11/0.255 | 1*/0.685 |
| II | aq. | 0.20/0.37/1.24 | 0.649/0.895/0.542 |
| | 0.5% NaCl | 0.10/0.26 | 0.978/0.73 |
| III | aq. | 0.03 | 0.86 |
| | 0.5% NaCl | 0.02 | 0.917 |

*indicates Newtonian flow behaviour

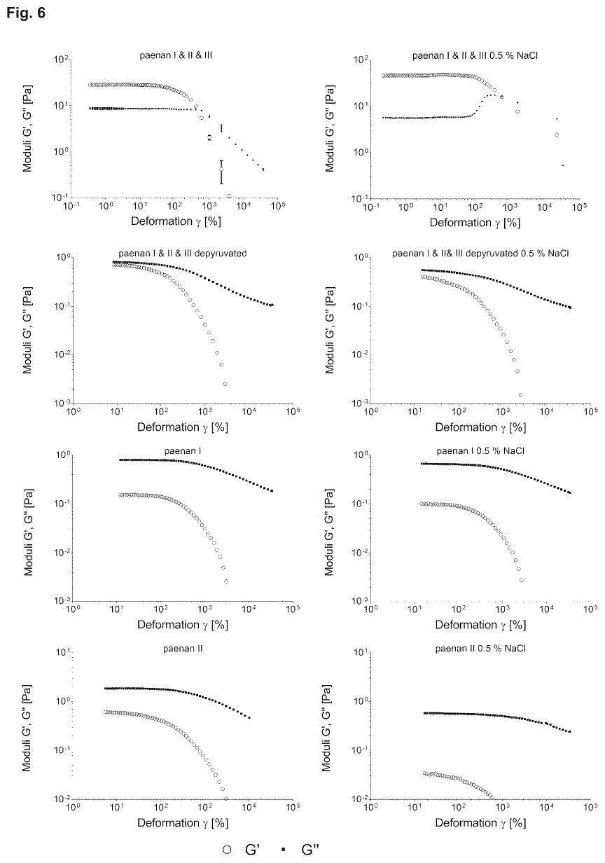

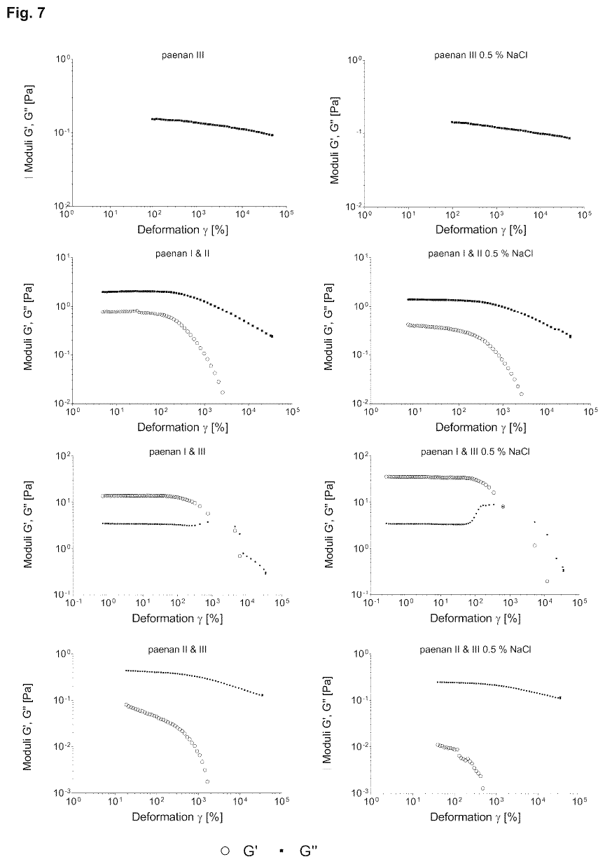

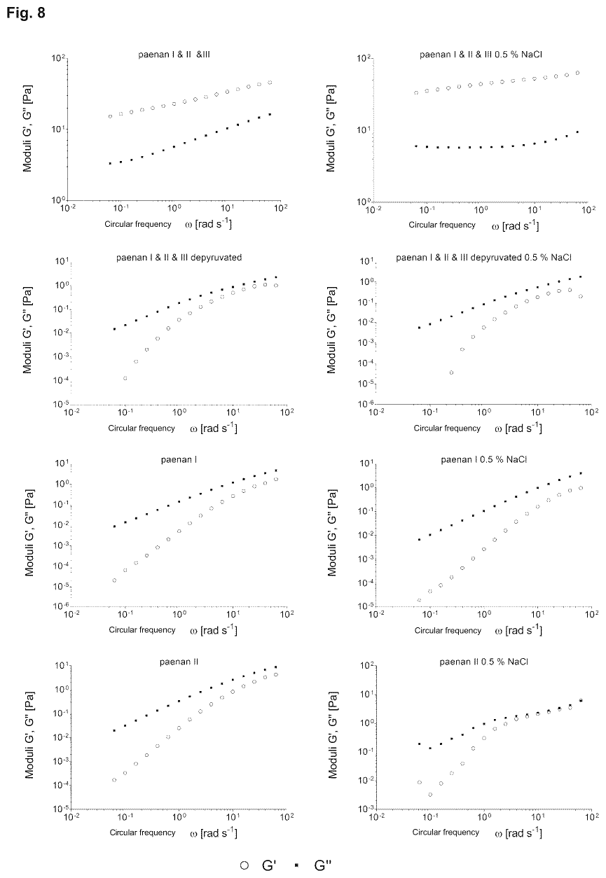

The basic viscoelastic properties determined by the amplitude sweep (FIGS. 6 and 7) are listed in Table 6. Paenan wild type (I & II & III) showed a soft and elastic gel-like behaviour with a yield strength of 9.1 Pa (32% elongation) and a yield point of 51.1 Pa (550% elongation) with a damping factor of 0.3 within the LVE. The addition of 0.5% NaCl led to an increased yield and an increased yield point of 32.3 Pa and 90.8 Pa, corresponding to an elongation of 82% and 590% respectively. A damping factor of 0.1 and the clear G″ peak after the LVE indicate a stronger but more brittle gel character. Frequency sweeps (FIG. 8) also showed a viscoelastic, liquid-like behaviour of G′ and G″ for paenan I & II & III with predominantly elastic behaviour in the entire frequency range analysed, which shifts more towards a gel-like behaviour with less frequency dependence of both G′ and G″ when NaCl is added. Both with and without the addition of NaCl, no crossover point was recognisable at low frequencies, which indicates long-term stability of the network.

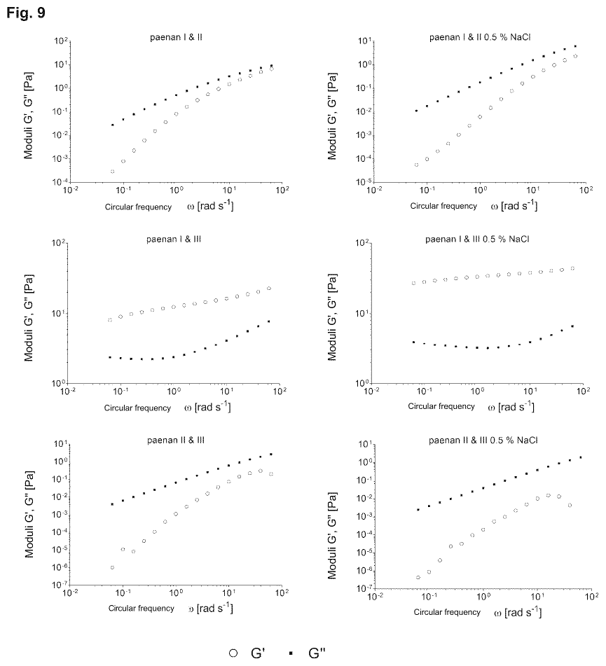

This gel character is most likely caused by the cation-mediated interactions between the pyruvyl residues of paenan I and the —COO— group of the glucuronic acid of paenan III. Further proof of this was provided by the depyruvylation of paenan I & II & III, which led to the complete loss of viscoelastic properties. In addition, all individual paenan polymers as well as the blends of paenan I & II and paenan II & III showed predominant fluid properties with Maxwell-like behaviour (FIGS. 8 and 9). Interestingly, with the exception of paenan II, no Maxwell fluid typical transition point could be observed at higher frequencies in the presence of NaCl, and the data indicated an incipient decrease in G′ at higher frequencies, leading to fluid behaviour at both low and high frequencies. This was particularly evident in paenan I & III.

The high viscosity and the pronounced intermolecular network, which leads to a gel-like character, make this polymer variant an interesting compound as a rheological thickening agent. Similar to other microbial polysaccharides, potential applications as rheology modifiers in food and beverages, but also technical applications such as oil drilling, appear promising. Compared to these polysaccharides, the viscosifying effect is greatly increased, suggesting that lower EPS concentrations are required to achieve similar results. In addition, the structurally related polysaccharide from P. polymyxa 2H2 has recently shown excellent compatibility with commonly used surfactants such as lauryl sulphate or cocamidopropyl betaine, which are typically used in cosmetics and personal care products. Consequently, the use of the wild-type EPS composition of P. polymyxa DSM 365 containing paenan I & II & III is suitable as a sustainable thickening agent for variable applications that can replace commercially available petroleum-based acrylic compounds.

TABLE 6
Viscoelastic properties of the paenan polymer variants.

| | | | | Yield | Yield |
|---|---|---|---|---|---|
| | | G′ [Pa] | tanδ | strength | point |
| Paenan variant | Solution | (LVE) | (LVE) | [Pa] | [Pa] |
| wt (I & | aq. | 28.5 | 0.3 | 9.1 | 51.1 |
| II & III) | 0.5% NaCl | 45.5 | 0.1 | 32.3 | 90.8 |
| wt (I & | aq. | 0.71 | 1.0 | n.d. | n.d. |
| II & III) | 0.5% NaCl | n.d. | 1.0 | n.d. | n.d. |
| depyruvylated | | | | | |
| I | aq. | 0.15 | 5.1 | n.d. | n.d. |
| | 0.5% NaCl | 0.10 | 6.7 | n.d. | n.d. |
| II | aq. | 0.60 | 3.0 | n.d. | n.d. |
| | 0.5% NaCl | n.d. | n.d. | n.d. | n.d. |
| III | aq. | n.d. | n.d. | n.d. | n.d. |
| | 0.5% NaCl | n.d. | n.d. | n.d. | n.d. |
| I & II | aq. | 0.78 | 2.6 | n.d. | n.d. |
| | 0.5% NaCl | 0.40 | 3.6 | n.d. | n.d. |
| I & III | aq. | 13.9 | 0.25 | 11 | 40 |
| | 0.5% NaCl | 35.8 | 0.1 | 18 | 79 |
| II & III | aq. | n.d. | n.d. | n.d. | n.d. |
| | 0.5% NaCl | n.d. | n.d. | n.d. | n.d. |

n.d.: not determined if measurement was not possible

The mixture of paenan I & III showed gel-like properties very similar to those of paenan I & II & III, indicating that the interaction is mainly between paenan I and paenan III. However, compared to paenan I & II & III, paenan I & III showed a lower gel strength with yield strengths at 13.9 Pa and 35.8 Pa with and without the presence of 0.5% NaCl and a less pronounced G″ peak at the end of the LVE region in the presence of NaCl. This indicates weaker interactions of these polymers. Analyses of the amplitude sweeps of the individual polymers showed that paenan I and II both exhibit viscoelastic, liquid-like behaviour, while paenan III only exhibits purely liquid behaviour. Without the formation of gel-like networks, the addition of NaCl led to a decrease in G′ and G″, while paenan I showed a higher salt stability compared to paenan II. This is also evident in the mixture of paenan I & II, where the effect of NaCl is more comparable to paenan I than to paenan II. These effects indicate an interaction between paenan I and II, which could be responsible for the increased gel strength of paenan I & II & III compared to paenan I & III. Since both paenan II and paenan III have a glucuronic acid in the backbone, the strong interaction of paenan I & III indicates a better accessibility of the glucuronic acid of paenan III compared to that of paenan II. In contrast, interactions between paenan II and paenan III could lead to a different structural arrangement of these polymers, resulting in better accessibility of the glucuronic acid in paenan II and thus increased interactions between paenan I & II in the wt. polymer mixture.

In contrast to the native polysaccharide composition, which contains paenan I & II & III, the deletion of individual polymers led to significantly altered viscoelastic properties. While the combination of paenan I & III still showed a distinct intermolecular network leading to a gel-like character, individual biopolymers showed a liquid-like behaviour, still forming films upon drying. Consequently, there are significantly different applications for the wild-type EPS composition. Thus, one application of the polysaccharides of the present invention is the formation of edible films and packaging materials similar to pullulan. On the other hand, the polysaccharides of the present invention can be used in high grade biomedical applications as coating materials in pharmaceutical systems for controlled drug release. For other charged polysaccharides such as hyaluronic acid and alginates, the chemical modification of the functional groups improved the targeting of specific cell types, enabling effective drug delivery systems. In addition, polysaccharides produced by other P. polymyxa strains have shown antioxidant activities that could further improve pharmacological applications.

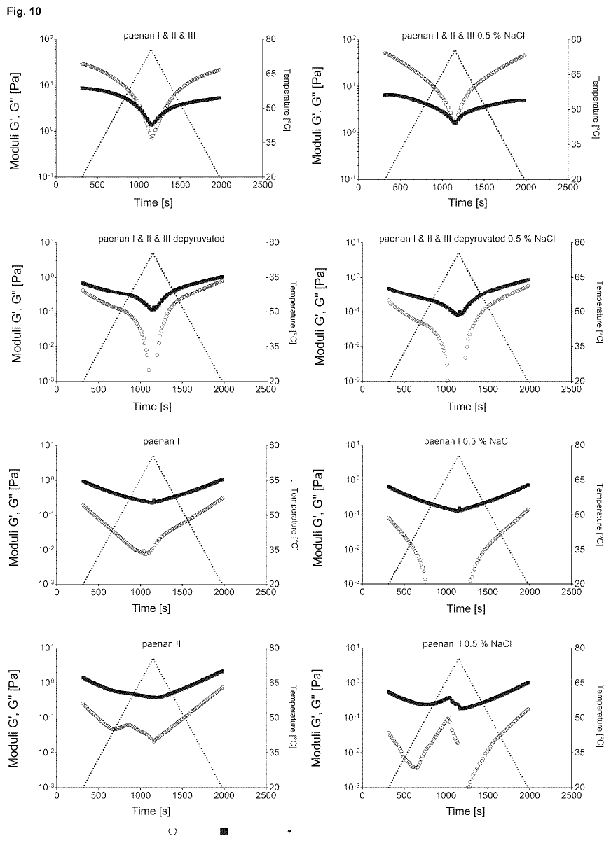

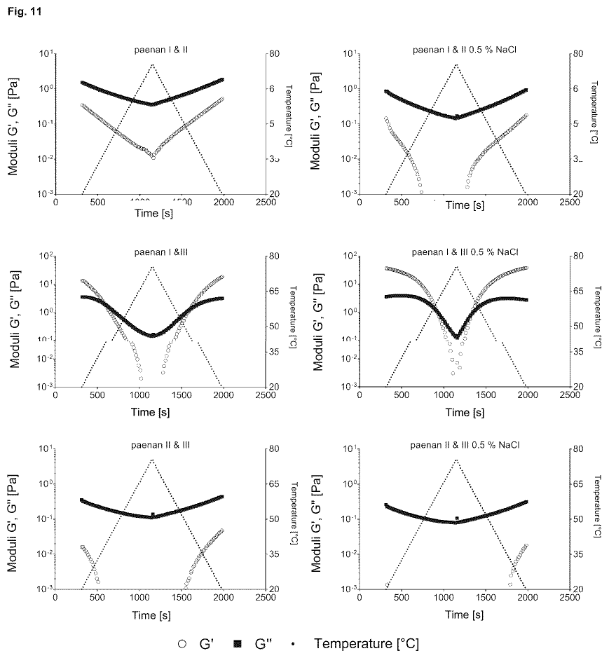

Temperature sweeps showed a high temperature dependence of the viscoelastic properties of paenan I & II & III both with and without the addition of NaCl (FIGS. 10 and 11). The viscoelastic properties of paenan I & III showed an even higher temperature dependence, which could be reduced by adding NaCl. While the native polysaccharide composition, which contained all three paenan variants, retained a weak gel character in the presence of NaCl up to 75° C., the deletion of paenan II led to a loss of temperature stability. This suggests a stabilising effect of paenan II with regard to the temperature stability of the polymer network, which is also evident from the high temperature stability of paenan II compared to paenan I & III. For paenan II, an increase in G′ and G″ was observed during the temperature increase, which was further intensified by the addition of NaCl. This effect is similar to that observed with the previously described undecorated xanthan variant and could also be explained by structural rearrangements of the individual polymer. However, these effects did not occur with any other combination of paenan II with other paenan polymers, suggesting a different arrangement of the individual polymers in the mixture, which further underscores the previously described differences between paenan I & II & III and paenan I & III in terms of their viscoelastic properties.

TABLE 7
Temperature stability of different paenan variants.

| | | | | relative gel | |
|---|---|---|---|---|---|
| Paenan | | G′ at | G′ at | strength at | tanδ at |
| variant | Solution | 20° C. | 75° C. | 75° C. [%] | 75° C. |
| wt (I & | aq | 29.43 | 1.15 | 3.91 | 1.49 |
| II & III) | 0.5% NaCl | 51.97 | 5.01 | 9.65 | 0.83 |
| wt (I & | aq. | 0.40 | 0.00 | 0.15 | n.d. |
| II & III) | 0.5% NaCl | 0.18 | 0.00 | 0.13 | n.d. |
| depyruvylated | | | | | |
| I | aq. | 0.17 | 0.01 | 6.19 | 22.29 |
| | 0.5% NaCl | 0.07 | 0.00 | 0.00 | n.d. |
| II | aq. | 0.269 | n.d. | n.d. | n.d. |
| | 0.5% NaCl | n.d. | n.d. | n.d. | n.d. |
| III | aq. | 0.139 | n.d. | n.d. | n.d. |
| | 0.5% NaCl | 0.139 | n.d. | n.d. | n.d. |
| I & II | aq. | 0.31 | 0.01 | 4.20 | 28.02 |
| | 0.5% NaCl | 0.08 | 0.00 | 0.00 | n.d. |
| I & III | aq. | 12.20 | 0.00 | 0.00 | n.d. |
| | 0.5% NaCl | 35.03 | 0.07 | 0.20 | 35.90 |
| II & III | aq. | 0.78 | 0.20 | 26.05 | 1.25 |
| | 0.5% NaCl | 0.78 | 0.12 | 15.71 | 1.23 |

n.d.: not determined if measurement in the LVE range was not possible

In addition, the thixotropic properties were determined by a three-stage oscillatory shear stress test (Table 8). While structural recovery was observed for all combinatorial variants of paenan, only 86.8% of the initial gel strength was measured for the wild-type EPS mixture after three minutes of non-destructive shear loading. This underscores a distinct intermolecular network that requires more time to recover and coordinate non-covalent interactions between individual polymers. Similar effects of delayed structural recovery were observed for polysaccharide compositions with paenan I & III, confirming the hypothesis that the gel-like character is mainly due to the cation-mediated interaction between the pyruvate of paenan I and the glucuronic acid residue of paenan III. In contrast, an immediate structural recovery was observed in all other knock-out variants, which led to the initial gel strength. Consequently, different variants could be applicable as binders that impart a thixotropic behaviour typically used for paints and coatings with different rheological profiles.

TABLE 8
Thixotropic recovery of paenan variants after a three-
stage oscillatory shear test. The thixotropic recovery
was calculated after 30/90/180 s based on G′ relative
to the initial values determined in the LVE range.

Thixotropic [%]
recovery

| Paenan variant | Solution | 30 s | 90 s | 180 s |
|---|---|---|---|---|
| Paenan | aq. | 79.3 | 83.5 | 85.6 |
| I & II & III | 0.5% NaCl | 82.2 | 84.7 | 86.8 |
| Paenan | aq. | n.d. | n.d. | n.d. |
| I & II & III | 0.5% NaCl | n.d. | n.d. | n.d. |
| (depyruvylated) | | | | |
| Paenan | aq. | 97.7 | 101.9 | 99.1 |
| I & II | 0.5% NaCl | 115.2 | 114.8 | 114.4 |
| Paenan | aq. | 81.9 | 91.6 | 96.4 |
| I & III | 0.5% NaCl | 106.4 | 110.7 | 112.2 |
| Paenan | aq. | 114.1 | 119.3 | 121.8 |
| II & III | 0.5% NaCl | 174.0 | 146.9 | 159.2 |
| Paenan | aq. | 103.2 | 103.4 | 103.5 |
| I | 0.5% NaCl | 107.2 | 109.6 | 108.1 |
| Paenan | aq. | n.d. | n.d. | n.d. |
| II | 0.5% NaCl | n.d. | n.d. | n.d. |
| Paenan | aq. | n.d. | n.d. | n.d. |
| III | 0.5% NaCl | n.d. | n.d. | n.d. |

n.d.: not determined for this variant due to limited LVE range

The rheological behaviour of heteroexopolysaccharides produced by P. polymyxa DSM 365 using CRISPR-Cas9-mediated knock-outs of glycosyltransferases was characterised. Viscoelastic properties of individual paenan variants and combinations thereof were analysed in detail. While the wild-type EPS composition showed a high viscosity and a gel-like behaviour, knock-out variants showed significantly altered physico-chemical properties depending on the individual polysaccharides present. Consequently, various polysaccharide compositions can be used for a wide range of applications, such as thickening agents or coating materials.

### Example 5—Mixing the Separately Prepared Polysaccharides and/or Polysaccharide Mixtures

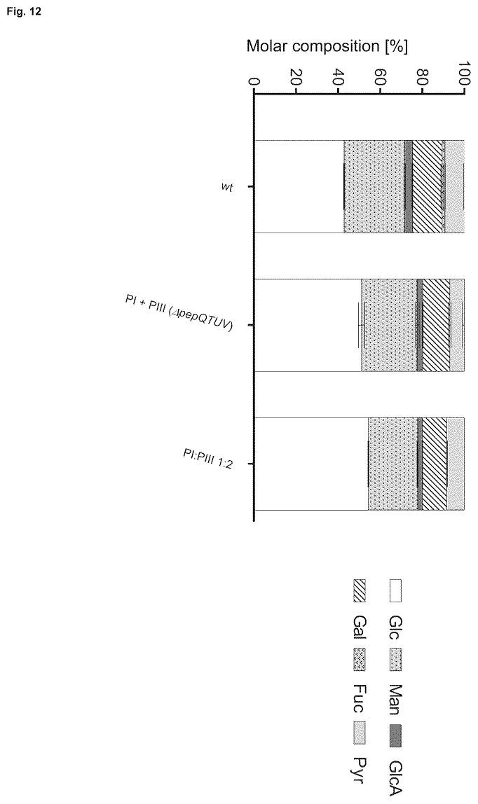

As shown in FIG. 12, both the monomer composition of the paenan wild type and its rheological properties could be restored by mixing the separately produced polysaccharides. The strong gel properties of the wild-type polymer composition are based on the interaction of the pyruvate group of paenan I (PI) and the GlcA of paenan III (PIII). By mixing the low-viscosity individual polymers PI and PIII in a ratio of 1:2, the naturally produced monomer composition of the deletion variant ΔpepQTUV can be recreated and the gel-forming and high-viscosity polymer properties restored (see also Table 8).

TABLE 8
shows the viscosities of exemplary polysaccharide mixtures
obtained by mixing separately prepared polysaccharides 1:2.

| | Total EPS | | |
|---|---|---|---|
| Polymer | concentration | Viscosity | |
| variant | [g/l] | [Pa s]* | tanδ** |
| WT | 10 | 26.773 | 0.241 |
| PI:PIII (1:2) | 10 | 26.567 | 0.158 |
| PI:PIII (1:2) | 15 | 48.349 | 0.172 |
| PI:PIII (1:2) | 20 | 79.569 | 0.163 |
| ΔpepI:PIII (1:2) | 10 | 16.932 | 0.243 |
| ΔpepI:PIII (1:2) | 15 | 36.164 | 0.248 |
| ΔpepI:PIII (1:2) | 20 | 51.519 | 0.252 |

*at a shear rate of 1.04/s
**in the linear viscoelastic range

ΔpepI refers to a mutation as a result of which the production organism carrying this mutation can no longer produce paenan III, but only paenan I and paenan II. By mixing with paenan III, a mixture can be produced that is similar to the wild type (WT). These results show that by selecting the individual polysaccharides or the polysaccharide mixtures, the mixing ratio and the total EPS concentration, a desired viscosity range can be set, which can correspond to the viscosity of the paenan wild type, but can also fall below or exceed it. This demonstrates flexibility and therefore versatility of application.

1. A method for producing a polysaccharide or a polysaccharide mixture by a production organism Paenibacillus polymyxa, wherein the method comprises at least one of the following steps separately from one another:
(a) production of the polysaccharide paenan I by a production organism Paenibacillus polymyxa, in which at least one enzymatic function of a polysaccharide biosynthesis cluster is switched off by genetic modification, so that neither the polysaccharide paenan II nor the polysaccharide paenan III is produced; and/or
(b) production of the polysaccharide paenan II by a production organism Paenibacillus polymyxa, in which at least one enzymatic function of a polysaccharide biosynthesis cluster is switched off by genetic modification, so that neither the polysaccharide paenan I nor the polysaccharide paenan III is produced; and/or
(c) production of the polysaccharide paenan III by a production organism Paenibacillus polymyxa, in which at least one enzymatic function of a polysaccharide biosynthesis cluster is switched off by genetic modification, so that neither the polysaccharide paenan I nor the polysaccharide paenan II is produced; and/or
(d) production of the polysaccharides paenan I and II by a production organism Paenibacillus polymyxa, in which at least one enzymatic function of a polysaccharide biosynthesis cluster is switched off by genetic modification, so that the polysaccharide paenan III is not produced; and/or
(e) production of the polysaccharides paenan II and III by a production organism Paenibacillus polymyxa, in which at least one enzymatic function of a polysaccharide biosynthesis cluster is switched off by genetic modification, so that the polysaccharide paenan I is not produced.

2. The method according claim 1, wherein the produced polysaccharide or polysaccharide mixture is purified after production; and/or wherein the production organism is Paenibacillus polymyxa DSM365.

3. The method according to claim 1, wherein the produced polysaccharides or polysaccharide mixtures are mixed, thereby obtaining a polysaccharide mixture comprising a mixture of the polysaccharides paenan I and paenan II, or a mixture of the polysaccharides paenan I and paenan III, or a mixture of the polysaccharides paenan II and paenan III, or a mixture of the polysaccharides paenan I, paenan II and paenan III.

4. The method according to claim 3, wherein paenan I and paenan III are mixed in a ratio of 1:2 m/v.

5. The method according to claim 1, wherein the genetic modification, by which at least one enzymatic function of a polysaccharide biosynthesis cluster is switched off so that the polysaccharide paenan I cannot be produced, is selected from genetic modifications which suppress the function of the glycosyltransferase PepD and/or PepF, and/or the undecaprenyl-glucose-phosphotransferase PepC, and/or wherein the genetic modification, by which at least one enzymatic function of a polysaccharide biosynthesis cluster is switched off so that the polysaccharide paenan II cannot be produced, is selected from genetic modifications which suppress the function of the glycosyltransferase PepT, PepU, and/or PepV, and/or the undecaprenyl-glucose phosphotransferase PepQ, and/or the GDP-L-fucose synthase; and/or wherein the genetic modification, by which at least one enzymatic function of a polysaccharide biosynthesis cluster is switched off so that the polysaccharide paenan III cannot be produced, is selected from genetic modifications which suppress the function of the glycosyltransferase PepI, PepJ, PepK, and/or PepL.

6. The method according to claim 1, wherein the genetic modification, by which at least one enzymatic function of a polysaccharide biosynthesis cluster is switched off, so that neither the polysaccharide paenan II nor the polysaccharide paenan III is produced, is selected from genetic modifications which switch off the function of the GDP-L-fucose synthase or the function of the glycosyltransferases PepI, PepT, PepU, and PepV, or the function of the glycosyltransferases PepK, PepT, PepU, and PepV, or the function of the glycosyltransferases PepL, PepT, PepU, and PepV, or the function of the glycosyltransferases PepT and PepL.

7. The method according to claim 1, wherein the genetic modification, by which at least one enzymatic function of a polysaccharide biosynthesis cluster is switched off, so that neither the polysaccharide paenan I nor the polysaccharide paenan III are produced, is selected from genetic modifications which suppress the function of the glycosyltransferases PepF and PepJ, or the function of the glycosyltransferases PepD and PepJ.

8. The method according to claim 1, wherein the genetic modification, by which at least one enzymatic function of a polysaccharide biosynthesis cluster is switched off, so that neither the polysaccharide paenan I nor the polysaccharide paenan II are produced, is selected from genetic modifications which suppress the function of the undecaprenyl-glucose phosphotransferase PepQ and the glycosyltransferase PepF.

9. A composition comprising the polysaccharide paenan I but neither the polysaccharide paenan II nor the polysaccharide paenan III; or
a composition comprising the polysaccharide paenan II but neither the polysaccharide paenan I nor the polysaccharide paenan III; or
a composition comprising the polysaccharide paenan III but neither the polysaccharide paenan I nor the polysaccharide paenan II; or
a composition comprising the polysaccharide paenan I and the polysaccharide paenan II but not the polysaccharide paenan III; or
a composition comprising the polysaccharide paenan II and the polysaccharide paenan III but not the polysaccharide paenan I.

10. A production organism Paenibacillus polymyxa, in which at least one enzymatic function of a polysaccharide biosynthesis cluster is switched off by genetic modification, so that neither the polysaccharide paenan II nor the polysaccharide paenan III can be produced; or
a production organism Paenibacillus polymyxa, in which at least one enzymatic function of a polysaccharide biosynthesis cluster is switched off by genetic modification, so that neither the polysaccharide paenan I nor the polysaccharide paenan III can be produced; or
a production organism Paenibacillus polymyxa, in which at least one enzymatic function of a polysaccharide biosynthesis cluster is switched off by genetic modification, so that neither the polysaccharide paenan I nor the polysaccharide paenan II can be produced; or
a production organism Paenibacillus polymyxa, in which at least one enzymatic function of a polysaccharide biosynthesis cluster is switched off by genetic modification, so that the polysaccharide paenan III cannot be produced; or
a production organism Paenibacillus polymyxa, in which at least one enzymatic function of a polysaccharide biosynthesis cluster is switched off by genetic modification, so that the polysaccharide paenan I cannot be produced.

11. A method of using the composition of claim 9; or a polysaccharide comprising paenan I, paenan II, or paenan III, or a mixture of any combination of paenan I, paenan II, and paenan III, wherein the composition or the polysaccharide is used as a rheological mediator, binder, stabilising agent, emulsifying agent, or flocculating agent, preferably in the food, pharmaceutical, and/or cosmetics sector.

12. The method according to claim 11, wherein the composition or the polysaccharide is used as an additive to a food product, optionally selected from bakery products, soups, sauces, mayonnaise, ketchup, jams, marmalades, jellies, canned foods (fruit and vegetables), salad dressing, ice cream, milk-based mixed drinks, puddings, pickles, meat, and jellies, ketchup, jams, marmalades, jellies, preserves (fruit and vegetables), salad dressings, ice cream, mixed milk drinks, puddings, pickled vegetables, tinned meat and fish, or as an additive in a cosmetic product, optionally selected from a shower gel, cosmetic creams and lotions, hair shampoo, skin creams, toothpaste, moisturisers, or as an additive in a pharmaceutical or medical product, optionally selected from wound dressings, controlled release particles, eye drops, tablet coatings, capsules for food supplements, or for use in tissue engineering to support surface adhesion after surgery, in water flooding for petroleum recovery, in bioremediation and wastewater treatment, for heavy metal binding, as a cement additive to keep particles in suspension during curing, or as a paint additive.


SEQUENCE LISTING (84 sequences)

SEQ ID NO: 1 - DNA, 24 bp, synthetic construct
  misc_feature 1..24: note = pepC_sgRNA1_fw
  source 1..24: mol_type = other DNA
acgcgagcatggaaaatttaccgg

SEQ ID NO: 2 - DNA, 24 bp, synthetic construct
  misc_feature 1..24: note = pepC_sgRNA1_rev
  source 1..24: mol_type = other DNA
aaacccggtaaattttccatgctc

SEQ ID NO: 3 - DNA, 24 bp, synthetic construct
  misc_feature 1..24: note = pepC_sgRNA2_fw
  source 1..24: mol_type = other DNA
aaacccttgatatgctgctgtcgt

SEQ ID NO: 4 - DNA, 24 bp, synthetic construct
  misc_feature 1..24: note = pepC_sgRNA_2_rev
  source 1..24: mol_type = other DNA
acgcacgacagcagcatatcaagg

SEQ ID NO: 5 - DNA, 43 bp, synthetic construct
  misc_feature 1..43: note = pepC_Harms_US_fw
  source 1..43: mol_type = other DNA
ggcctctagaccaaaattcgatgaagcaaggagaatgagtgcg

SEQ ID NO: 6 - DNA, 54 bp, synthetic construct
  misc_feature 1..54: note = pepC_Harms_US_rev_OE
  source 1..54: mol_type = other DNA
cgcttgaatgcatccatcaataagcatcccgtaccccctctcaacctgccgtgg

SEQ ID NO: 7 - DNA, 54 bp, synthetic construct
  misc_feature 1..54: note = pepC_Harms_DS_fw_OE
  source 1..54: mol_type = other DNA
ccacggcaggttgagagggggtacgggatgcttattgatggatgcattcaagcg

SEQ ID NO: 8 - DNA, 36 bp, synthetic construct
  misc_feature 1..36: note = pepC_Harms_DS_rev
  source 1..36: mol_type = other DNA
ggcctctagaccatctttcaccccgcagctgactcg

SEQ ID NO: 9 - DNA, 28 bp, synthetic construct
  misc_feature 1..28: note = pepC_KO_proof_fw
  source 1..28: mol_type = other DNA
gcaactcatggagcaggccagtgcgacg

SEQ ID NO: 10 - DNA, 31 bp, synthetic construct
  misc_feature 1..31: note = pepC_KO_proof_rev
  source 1..31: mol_type = other DNA
gctgtatggtgtttattctagtagtccaagg

SEQ ID NO: 11 - DNA, 24 bp, synthetic construct
  misc_feature 1..24: note = pepD_sgRNA1_fw
  source 1..24: mol_type = other DNA
acgccaaagtgttatacatcacgg

SEQ ID NO: 12 - DNA, 24 bp, synthetic construct
  misc_feature 1..24: note = pepD_sgRNA1_rev
  source 1..24: mol_type = other DNA
aaacccgtgatgtataacactttg

SEQ ID NO: 13 - DNA, 24 bp, synthetic construct
  misc_feature 1..24: note = pepD_sgRNA2_fw
  source 1..24: mol_type = other DNA
acgcatgaagcgggaacataacgg

SEQ ID NO: 14 - DNA, 24 bp, synthetic construct
  misc_feature 1..24: note = pepD_sgRNA_2_rev
  source 1..24: mol_type = other DNA
aaacccgttatgttcccgcttcat

SEQ ID NO: 15 - DNA, 32 bp, synthetic construct
  misc_feature 1..32: note = pepD_Harms_US_fw
  source 1..32: mol_type = other DNA
ccggtctagaggctattatgggtcgctacgtg

SEQ ID NO: 16 - DNA, 50 bp, synthetic construct
  misc_feature 1..50: note = pepD_Harms_US_rev_OE
  source 1..50: mol_type = other DNA
agtccaaggatagttactcatgttcccaataagcatccttggagaacagc

SEQ ID NO: 17 - DNA, 46 bp, synthetic construct
  misc_feature 1..46: note = pepD_Harms_DS_fw_OE
  source 1..46: mol_type = other DNA
ttctccaaggatgcttattgggaacatgagtaactatccttggact

SEQ ID NO: 18 - DNA, 30 bp, synthetic construct
  misc_feature 1..30: note = pepD_Harms_DS_rev
  source 1..30: mol_type = other DNA
aatttctagacgttaccgcgccaagacctc

SEQ ID NO: 19 - DNA, 20 bp, synthetic construct
  misc_feature 1..20: note = pepD_KO_proof_fw
  source 1..20: mol_type = other DNA
ccggatcagcaggacggacg

SEQ ID NO: 20 - DNA, 20 bp, synthetic construct
  misc_feature 1..20: note = pepD_KO_proof_rev
  source 1..20: mol_type = other DNA
gcccgcaaccgatatatccc

SEQ ID NO: 21 - DNA, 24 bp, synthetic construct
  misc_feature 1..24: note = pepI_sgRNA1_fw
  source 1..24: mol_type = other DNA
acgcagcattatgagctaaccaag

SEQ ID NO: 22 - DNA, 24 bp, synthetic construct
  misc_feature 1..24: note = pepI_sgRNA1_rev
  source 1..24: mol_type = other DNA
aaaccttggttagctcataatgct

SEQ ID NO: 23 - DNA, 32 bp, synthetic construct
  misc_feature 1..32: note = pepI_Harms_US_fw
  source 1..32: mol_type = other DNA
aatttctagaggctgcgctctttttcttcatc

SEQ ID NO: 24 - DNA, 37 bp, synthetic construct
  misc_feature 1..37: note = pepI_Harms_US_rev_OE
  source 1..37: mol_type = other DNA
gcaatacgaaatccgtagcgggtgcctccttctttat

SEQ ID NO: 25 - DNA, 36 bp, synthetic construct
  misc_feature 1..36: note = pepI_Harms_DS_fw_OE
  source 1..36: mol_type = other DNA
aggaggcacccgctacggatttcgtattgcggcaac

SEQ ID NO: 26 - DNA, 30 bp, synthetic construct
  misc_feature 1..30: note = pepI_Harms_DS_rev
  source 1..30: mol_type = other DNA
ttaatctagaggcttcactgtccgttccct

SEQ ID NO: 27 - DNA, 21 bp, synthetic construct
  misc_feature 1..21: note = pepI_KO_proof_fw
  source 1..21: mol_type = other DNA
gttgctggctaccatgttcgg

SEQ ID NO: 28 - DNA, 24 bp, synthetic construct
  misc_feature 1..24: note = pepI_KO_proof_rev
  source 1..24: mol_type = other DNA
ccttgcattcggtagaagttcacg

SEQ ID NO: 29 - DNA, 24 bp, synthetic construct
  misc_feature 1..24: note = pepK_sgRNA2_fw
  source 1..24: mol_type = other DNA
acgcacaatcggcttcagatacgg

SEQ ID NO: 30 - DNA, 24 bp, synthetic construct
  misc_feature 1..24: note = pepK_sgRNA_2_rev
  source 1..24: mol_type = other DNA
aaacccgtatctgaagccgattgt

SEQ ID NO: 31 - DNA, 31 bp, synthetic construct
  misc_feature 1..31: note = pepK_Harms_US_fw
  source 1..31: mol_type = other DNA
aatttctagacatacgattggcagcaagcag

SEQ ID NO: 32 - DNA, 39 bp, synthetic construct
  misc_feature 1..39: note = pepK_Harms_US_rev_OE
  source 1..39: mol_type = other DNA
tttaacgcgaatgtcggtttcgccgccgcctttctgtca

SEQ ID NO: 33 - DNA, 39 bp, synthetic construct
  misc_feature 1..39: note = pepK_Harms_DS_fw_OE
  source 1..39: mol_type = other DNA
aaaggcggcggcgaaccgacattcgcgttaaaggatgat

SEQ ID NO: 34 - DNA, 32 bp, synthetic construct
  misc_feature 1..32: note = pepK_Harms_DS_rev
  source 1..32: mol_type = other DNA
aatttctagagctgctcgacctttgcgacggc

SEQ ID NO: 35 - DNA, 24 bp, synthetic construct
  misc_feature 1..24: note = pepK_KO_proof_fw
  source 1..24: mol_type = other DNA
gctctaatctgtagtgacgagcag

SEQ ID NO: 36 - DNA, 21 bp, synthetic construct
  misc_feature 1..21: note = pepK_KO_proof_rev
  source 1..21: mol_type = other DNA
catcaaatttgcgggcatggg

SEQ ID NO: 37 - DNA, 24 bp, synthetic construct
  misc_feature 1..24: note = pepL_sgRNA1_fw
  source 1..24: mol_type = other DNA
acgcggaggctcctatcttcacaa

SEQ ID NO: 38 - DNA, 24 bp, synthetic construct
  misc_feature 1..24: note = pepL_sgRNA1_rev
  source 1..24: mol_type = other DNA
aaacttgtgaagataggagcctcc

SEQ ID NO: 39 - DNA, 24 bp, synthetic construct
  misc_feature 1..24: note = pepL_sgRNA2_fw
  source 1..24: mol_type = other DNA
acgcgttattgtcacacaccgatg

SEQ ID NO: 40 - DNA, 24 bp, synthetic construct
  misc_feature 1..24: note = pepL_sgRNA_2_rev
  source 1..24: mol_type = other DNA
aaaccatcggtgtgtgacaataac

SEQ ID NO: 41 - DNA, 30 bp, synthetic construct
  misc_feature 1..30: note = pepL_Harms_US_fw
  source 1..30: mol_type = other DNA
aatttctagagcgcatcgttccattggacg

SEQ ID NO: 42 - DNA, 43 bp, synthetic construct
  misc_feature 1..43: note = pepL_Harms_US_rev_OE
  source 1..43: mol_type = other DNA
ccagcttcattccgtctaaatcatcctttaacgcgaatgtcgg

SEQ ID NO: 43 - DNA, 38 bp, synthetic construct
  misc_feature 1..38: note = pepL_Harms_DS_fw_OE
  source 1..38: mol_type = other DNA
taaaggatgatttagacggaatgaagctggctgtaatt

SEQ ID NO: 44 - DNA, 30 bp, synthetic construct
  misc_feature 1..30: note = pepL_Harms_DS_rev
  source 1..30: mol_type = other DNA
aatttctagagcgcctgcctgaatgagtgt

SEQ ID NO: 45 - DNA, 22 bp, synthetic construct
  misc_feature 1..22: note = pepL_KO_proof_fw
  source 1..22: mol_type = other DNA
cggagaacatatcgacccgctc

SEQ ID NO: 46 - DNA, 22 bp, synthetic construct
  misc_feature 1..22: note = pepL_KO_proof_rev
  source 1..22: mol_type = other DNA
ggccagttgtacgagatcgacc

SEQ ID NO: 47 - DNA, 24 bp, synthetic construct
  misc_feature 1..24: note = pepT_sgRNA1_fw
  source 1..24: mol_type = other DNA
acgcagaacgtgaggaaaaccgtg

SEQ ID NO: 48 - DNA, 24 bp, synthetic construct
  misc_feature 1..24: note = pepT_sgRNA1_rev
  source 1..24: mol_type = other DNA
aaaccacggttttcctcacgttct

SEQ ID NO: 49 - DNA, 24 bp, synthetic construct
  misc_feature 1..24: note = pepT_sgRNA2_fw
  source 1..24: mol_type = other DNA
acgctatgtcgtgggatcgcattg

SEQ ID NO: 50 - DNA, 24 bp, synthetic construct
  misc_feature 1..24: note = pepT_sgRNA_2_rev
  source 1..24: mol_type = other DNA
aaaccaatgcgatcccacgacata

SEQ ID NO: 51 - DNA, 31 bp, synthetic construct
  misc_feature 1..31: note = pepT_Harms_US_fw
  source 1..31: mol_type = other DNA
aatttctagacccggatcatcgctttacagt

SEQ ID NO: 52 - DNA, 35 bp, synthetic construct
  misc_feature 1..35: note = pepT_Harms_US_rev_OE
  source 1..35: mol_type = other DNA
tgctgacctgttggcggggtttagagaccgctgcg

SEQ ID NO: 53 - DNA, 35 bp, synthetic construct
  misc_feature 1..35: note = pepT_Harms_DS_fw_OE
  source 1..35: mol_type = other DNA
ctgaagaagcgctaggccaacaggtcagcagacag

SEQ ID NO: 54 - DNA, 31 bp, synthetic construct
  misc_feature 1..31: note = pepT_Harms_DS_rev
  source 1..31: mol_type = other DNA
aatttctagagtcctccacgacttcctccag

SEQ ID NO: 55 - DNA, 20 bp, synthetic construct
  misc_feature 1..20: note = pepT_KO_proof_fw
  source 1..20: mol_type = other DNA
cgcagctcaaacgtgggcta

SEQ ID NO: 56 - DNA, 20 bp, synthetic construct
  misc_feature 1..20: note = pepT_KO_proof_rev
  source 1..20: mol_type = other DNA
gggctactgcacagcgaatc

SEQ ID NO: 57 - DNA, 24 bp, synthetic construct
  misc_feature 1..24: note = pepU_sgRNA1_fw
  source 1..24: mol_type = other DNA
acgcagaaaccgaacatcttcctg

SEQ ID NO: 58 - DNA, 24 bp, synthetic construct
  misc_feature 1..24: note = pepU_sgRNA1_rev
  source 1..24: mol_type = other DNA
aaaccaggaagatgttcggtttct

SEQ ID NO: 59 - DNA, 24 bp, synthetic construct
  misc_feature 1..24: note = pepU_sgRNA2_fw
  source 1..24: mol_type = other DNA
acgctttaccgcgatacgatccag

SEQ ID NO: 60 - DNA, 24 bp, synthetic construct
  misc_feature 1..24: note = pepU_sgRNA_2_rev
  source 1..24: mol_type = other DNA
aaacctggatcgtatcgcggtaaa

SEQ ID NO: 61 - DNA, 35 bp, synthetic construct
  misc_feature 1..35: note = pepU_Harms_US_fw
  source 1..35: mol_type = other DNA
aatttctagaggatgcttatggtctggtcattact

SEQ ID NO: 62 - DNA, 32 bp, synthetic construct
  misc_feature 1..32: note = pepU_Harms_US_rev_OE
  source 1..32: mol_type = other DNA
cacattcacttcctgtctgctgacctgttggc

SEQ ID NO: 63 - DNA, 33 bp, synthetic construct
  misc_feature 1..33: note = pepU_Harms_DS_fw_OE
  source 1..33: mol_type = other DNA
cagcagacaggaagtgaatgtgaagcagctgat

SEQ ID NO: 64 - DNA, 34 bp, synthetic construct
  misc_feature 1..34: note = pepU_Harms_DS_rev
  source 1..34: mol_type = other DNA
aatttctagagcacgcagctgatgaattttatcc

SEQ ID NO: 65 - DNA, 21 bp, synthetic construct
  misc_feature 1..21: note = pepU_KO_proof_fw
  source 1..21: mol_type = other DNA
gcgtattatctggctccacgg

SEQ ID NO: 66 - DNA, 24 bp, synthetic construct
  misc_feature 1..24: note = pepU_KO_proof_rev
  source 1..24: mol_type = other DNA
gttgtaaagcttagtcggccttgt

SEQ ID NO: 67 - DNA, 24 bp, synthetic construct
  misc_feature 1..24: note = pepV_sgRNA1_fw
  source 1..24: mol_type = other DNA
acgcttaccctggaatccgcatcg

SEQ ID NO: 68 - DNA, 24 bp, synthetic construct
  misc_feature 1..24: note = pepV_sgRNA1_rev
  source 1..24: mol_type = other DNA
aaaccgatgcggattccagggtaa

SEQ ID NO: 69 - DNA, 36 bp, synthetic construct
  misc_feature 1..36: note = pepV_Harms_US_fw
  source 1..36: mol_type = other DNA
aatttctagaaagaatcaagaagctgcttttacagg

SEQ ID NO: 70 - DNA, 31 bp, synthetic construct
  misc_feature 1..31: note = pepV_Harms_US_rev_OE
  source 1..31: mol_type = other DNA
tttaccgactgcatccgcaatccttccaact

SEQ ID NO: 71 - DNA, 32 bp, synthetic construct
  misc_feature 1..32: note = pepV_Harms_DS_fw_OE
  source 1..32: mol_type = other DNA
attgcggatgcagtcggtaaaagcaggatgca

SEQ ID NO: 72 - DNA, 32 bp, synthetic construct
  misc_feature 1..32: note = pepV_Harms_DS_rev
  source 1..32: mol_type = other DNA
aatttctagagccagtccccatataccaatcg

SEQ ID NO: 73 - DNA, 20 bp, synthetic construct
  misc_feature 1..20: note = pepV_KO_proof_fw
  source 1..20: mol_type = other DNA
agcgaacatggcaaagctgg

SEQ ID NO: 74 - DNA, 21 bp, synthetic construct
  misc_feature 1..21: note = pepV_KO_proof_rev
  source 1..21: mol_type = other DNA
ggggcatcccttacgtcatct

SEQ ID NO: 75 - DNA, 24 bp, synthetic construct
  misc_feature 1..24: note = fcl_sgRNA1_fw
  source 1..24: mol_type = other DNA
acgcgatacatctaccaacttacg

SEQ ID NO: 76 - DNA, 24 bp, synthetic construct
  misc_feature 1..24: note = fcl_sgRNA1_rev
  source 1..24: mol_type = other DNA
aaaccgtaagttggtagatgtatc

SEQ ID NO: 77 - DNA, 24 bp, synthetic construct
  misc_feature 1..24: note = fcl_sgRNA2_fw
  source 1..24: mol_type = other DNA
acgcacaaacgaatgtgattgatg

SEQ ID NO: 78 - DNA, 24 bp, synthetic construct
  misc_feature 1..24: note = fcl_sgRNA2_rev
  source 1..24: mol_type = other DNA
aaaccatcaatcacattcgtttgt

SEQ ID NO: 79 - DNA, 36 bp, synthetic construct
  misc_feature 1..36: note = fcl_US_harms_fw
  source 1..36: mol_type = other DNA
aattaatctagagacgtgatcttaatggatacgccg

SEQ ID NO: 80 - DNA, 34 bp, synthetic construct
  misc_feature 1..34: note = fcl_US_harms_rev_OE
  source 1..34: mol_type = other DNA
ccaccgttttctttaatacgcatccttggagcct

SEQ ID NO: 81 - DNA, 29 bp, synthetic construct
  misc_feature 1..29: note = fcl_DS_harms_fw_OE
  source 1..29: mol_type = other DNA
cgtattaaagaaaacggtgggggaattgg

SEQ ID NO: 82 - DNA, 32 bp, synthetic construct
  misc_feature 1..32: note = fcl_DS_harms_rev
  source 1..32: mol_type = other DNA
aatttctagatctcttacacgacgacagaagc

SEQ ID NO: 83 - DNA, 20 bp, synthetic construct
  misc_feature 1..20: note = fcl_Koproof_fw
  source 1..20: mol_type = other DNA
aatccgtccgagatgctcag

SEQ ID NO: 84 - DNA, 20 bp, synthetic construct
  misc_feature 1..20: note = fcl_Koproof_rev
  source 1..20: mol_type = other DNA
ccttgcgtacagcgaagaga